(19) United States
(12) Patent Application Publication
(10) Pub. No.: US 2010/0260770 A1
(43) Pub. Date: Oct. 14, 2010

(54) IL-33 IN INFLAMMATORY DISEASE

(75) Inventor: Anthony Coyle, Washington, DC (US)

Correspondence Address: MEDIMMUNE, LLC;Patrick Scott Alban, ONE MEDIMMUNE WAY, GAITHERSBURG, MD, 20878, US

(73) Assignee: MEDIMMUNE, LLC, Gaithersburg, MD (US)

(21) Appl. No.: 12/600,548

(22) PCT Filed: May 19, 2008
(86) PCT No.: PCT/US08/64048

§ 371 Date: Jun. 9, 2010

Related U.S. Application Data
(60) Provisional application No. 60/924,542, filed on May 18, 2007.
(60) Provisional application No. 61/064,167, filed on Feb. 20, 2008.

(51) Int. Cl. A61K 39/395; A61K 38/00; C07K 16/24; C07K 14/00

(52) U.S. Cl. 424/158.1; 514/1.1; 530/387.1; 530/350

(57) ABSTRACT

The present invention encompasses IL-33 specific binding polypeptides and compositions comprising IL-33 specific binding polypeptides, e.g., antibodies and monomer/multimer domain polypeptides. The invention also encompasses methods employing the IL-33 specific binding polypeptides to treat diseases and disorders such as asthma.

### FIELD OF THE INVENTION

The present invention relates to IL-33 and inflammatory disease.

### BACKGROUND OF THE INVENTION

The present invention encompasses IL-33 specific binding polypeptides and compositions comprising the IL-33 specific binding polypeptides. The IL-33 specific binding polypeptides may be antibodies, monomer domain polypeptides, or multimer domain polypeptides. These polypeptides and the compositions comprising these polypeptides are useful in treating inflammatory diseases, e.g., asthma.

### SUMMARY OF THE INVENTION

One embodiment of the invention encompasses a method of treating an inflammatory disorder. An IL-33 specific binding composition is administered to a subject. The IL-33 specific binding composition may comprise an antibody, a monomer domain polypeptide, or a multimer domain polypeptide.

Another embodiment of the invention encompasses a composition comprising an IL-33 specific polypeptide. The IL-33 specific polypeptide is an antibody, a monomer domain polypeptide, or a multimer domain polypeptide.

### BRIEF DESCRIPTION OF THE FIGURES

FIG. 1A-D: IL-33 is a potent activator of mouse BMMCs, as measured by IL-6 production. (A) IL-33 induces a potent response compared to other stimuli, e.g., IgE receptor cross-linking or LPS; (B) T1/ST2 antibody inhibits IL-6 production by IL-33 stimulated cells; (C) IL-33 appears to synergize with IgE receptor cross-linking but apparently has no effect in the presence of TLR ligands; (D) IL-33 induced cytokine production is MyD88 dependent; IL-33 has no effect on BMMCs from MyD88 deficient mice. Results are expressed as means±SEM.

FIG. 2A-B: IL-33 activation induces various inflammatory mediators in addition to IL-6. (A) Induced cytokines and chemokines by IL-33 alone (red) or in synergy with IgE receptor cross-linking (blue), ↑ indicates induction and [symbol] indicates no induction (B) time dependent induction of cysteinyl leukotrienes by IL-33 activation. Results are expressed as means±SEM.

FIG. 3: IL-33 does not appear to induce degranulation. Results are expressed as means ±SEM.

FIG. 4A-D: IL-33 induces AHR in naive mice. BALB/c mice were treated intranasally (i.n.) with either IL-33 (red lines), IL-13 (blue lines) or PBS (black lines). AHR was assessed 4 or 24 h later. (A) methacholine dose-response curves for Penh at 4 hours; (B) methacholine dose-response curves for Penh at 24 hours; (C), airways resistance; and (D) compliance. Results are expressed as the means±SEM (PBS n=6, IL-13 & IL-33 n=8 mice/group). Significant differences between respective PBS and IL-33 treated mice and between PBS and IL-13 treated mice are indicated as *P<0.05-P<0.01.

FIG. 5A-D: IL-33 induces mRNA expression of IL-5, IL-13 and Mucin genes Gob-5 and MUC5AC in mouse lung. IL-33 induces significant expression of (A) IL-5; (B) IL-13; (C) GOB5; and (D) MUC5AC. IL-13 significantly upregulates mucin genes, (C) GOB5; and (D) MUC5AC. Results are expressed as the means±SEM (PBS n=6, IL-13 & IL-33 n=8 mice/group). Significant differences between respective PBS and IL-33 treated mice and between PBS and IL-13 treated mice are indicated as *P<0.05-P<0.01.

FIG. 6A-B: IL-33 directly activates Mast Cells as shown by induction of mMCP-1. IL-33 induced a significant up-regulation of mMCP-1 in both (A) lung tissue; and (B) serum while IL-13 had no such effect. Results are expressed as the means±SEM (PBS n=6, IL-13 & IL-33 n=8 mice/group). Significant differences between respective PBS and IL-33 treated mice and between PBS and IL-13 treated mice are indicated as *P<0.05-P<0.01

FIG. 7A-E: IL-33 activates Human Cord Blood Derived Mast Cells (HMCs). HMC supernatants from untreated (open bars), IL-33 stimulated (red bars), IgE receptor cross-linking (black bars) and IL-33+IgE receptor cross-linking (blue bars) were taken at the specific time points and assayed by ELISA. IL-33 stimulation of HMCs induced the production of (A) IL-5 and (B) IL-13 when compared to untreated cells. Interestingly, IL-33+IgE receptor cross-linking significantly enhanced the production of (A) IL-5, (B) IL-13, and (C) TNF-alpha from these cells. IL-33 also induced (D) PGD2 and (E) PGE2 production. Results are expressed as the means±SEM.

FIGS. 8a-8f: IL-33 is a potent activator of mouse and human mast cells. BMMC were stimulated with a four point dose response curve of IL-33 (0.1-100 ng/ml) for 6 and 24 h. BMMC supernatants were then analyzed at 24 h for IL-6 (a; open columns), IL-13 (a; filled columns), CysLT (b; open columns), and at 6 h for PGD2 (b; filled columns). (c) IL-33 mediates IL-6 production in a MyD88 dependent but TRIF independent manner. BMMC from wildtype, MyD88^{-/-}, or TRIF^{-/-} BMMC were stimulated by IL-33 (10 ng/ml) or IL-1b (10 ng/ml), or in combination with IgER crosslinking (CL). After 24 h of stimulation, levels of IL-6 were assessed by ELISA. (d) BMMC supernatants were assessed for mouse mast cell protease-1 (mMCP-1) levels after 90 m of stimulation with IL-33 (0.1-100 ng/ml) or IgER crosslinking (e) IL-33 appears to synergize with IgE Receptor crosslinking BMMC were synergistically activated with IL-33 (0.1-100 ng/ml) in combination with IgER crosslinking Supernatants were then analyzed for IL-6 (open columns), IL-13 (filled columns), and CysLT (hatched columns). (f) HMC were stimulated (open columns) or not (filled columns) with IL-33 (10 ng/ml) for 24 h. HMC supernatants were then analyzed for IL-5, IL-13, CysLT, and PGD2. HMC results are representative of two independent experiments obtained with different batches of cells. BMMC results are representative of three independent experiments obtained with different batches of cells. All data are expressed as means±SEM.

FIGS. 9a-9e: Administration of IL-33 induced AHR in naïve mice. One (solid triangles) or three doses (solid circles) of recombinant IL-33 or PBS (solid squares) was delivered intranasally to naïve BALB/c mice. AHR was assessed in anaethetised and tracheostomised mice by measuring changes in lung resistance (a), elastance (b), tissue resistance (G; c), tissue elastance (H; d) and Newtonian resistance of the airways (Rₙ, e), in response to increasing concentrations of methacholine after 1 day and 3 days using the flexivent system. Data are expressed as mean±SEM, n=6-11 mice/group; *p<0.05 (two-way ANOVA) in comparison to PBS treated mice.

FIGS. 10a-10f: Administration of IL-33 induced Th2 type inflammation in naïve mice. One (hatched bars), two (striped bars) or three (solid bars) doses of IL-33 or PBS (open bars) was delivered intranasally to naïve mice, and lung inflammation was assessed at 1, 2 and 3 days post-administration. Leukocytes were isolated from the airway lumen (a; total, eo, mac, neut:*p<0.0001; lymph:*p=0.0014) and lung tissue (b; total, eo, mac, neut:*p<0.0001; lymph: *p=0.0003) and phenotyped. Expression of the mucin-related genes MUC5ac and Gob-5 (c) was assessed in the lung tissue by Taqman and expressed relative to GAPDH (MUC5ac 1 dose:*p=0.003; 2 and 3 doses:*p<0.0001; Gob-5: *p<0.0001). Numbers of CD4 T cells (d; 1 dose:*p=0.0486; 2 doses:*p=0.0006; 3 doses:*p<0.0001) and CD4/T1/ST2 T cells (e; 1 dose:*p=0.006; 2 doses:*p=0.0001; 3 doses:*p<0.0001) were quantified by flow cytometric analysis. Mast cell activation (f; 1 dose:*p=0.0212; 2 and 3 doses:*p<0.0001) was determined by quantifying levels of mMCP-1 in serum. Data are expressed as mean±SEM, n=8-30 mice/group; *p<0.05 (Mann-Whitney U test) in comparison to PBS treated mice.

FIGS. 11a-11f: Induction of AHR was not dependent on CD4 cells. Mice were treated with a depleting antibody against CD4 prior to IL-33 administration, and AHR and inflammation were assessed 3 days later. Total CD4 depletion was confirmed by flow cytometric analysis (a-b; CD4:*p=0.0043, Mann-Whitney U test; CD4/T1ST2:*p=0.0022, Mann-Whitney U test). AHR was assessed by measuring changes in resistance (c; *p<0.05; ^{#}p<0.05, two-way ANOVA) and elastance (d; p<0.05; ^{#}p<0.05, two-way ANOVA) in response to increasing concentrations of methacholine. Leukocyte recruitment to the lung tissue (e; Total:*p=0.0022, ^{#}p=0.0002; Eo:*p=0.0022, ^{#}p=0.0002; Mac:*p=0.0260, ^{#}p=0.0003; Lymph:*p=0.0649, ^{#}p=0.0002; Neut:*p=0.0022, ^{#}p=0.0003, Mann-Whitney U test) was assessed as previously described. Mast cell activation (f) was determined by quantifying serum levels of mMCP-1 (*p=0.0009, ¹⁹⁰ p=0.0001, Mann-Whitney U test). Data are expressed as mean±SEM, n=6-18 mice/group; *p<0.05 IL-33+Ig treated mice (solid circles/solid bars) in comparison to PBS+Ig treated mice (open bars/solid squares); ^{#}p<0.05 IL-33+anti-CD4 treated mice (slashed bars/open circles) in comparison to PBS+anti-CD4 treated mice (grey bars/open squares).

FIGS. 12a-12e: Mast cell deficient mice were protected from IL-33-induced AHR. IL-33 was delivered intranasally to wild type and mast cell deficient (Kit^{W-sh/W-sh}) mice, and AHR and inflammation assessed 3 days later. AHR was assessed by measuring changes in resistance (a; *p<0.001, two-way ANOVA) and elastance (b; *p<0.01, ^{#}p<0.01, two-way ANOVA) in response to increasing concentrations of methacholine. Expression of IL-13 (c; *p=0.0111, ^{#}p=0.0006, Mann-Whitney U test), and PGD₂ and leukotrienes (d; PGD₂:*p<0.0001, ^{#}p=0.0012; LTs:*p<0.0001, ^{#}p<0.0255, Mann-Whitney U test) was determined in BAL supernatent by ELISA. Serum mMCP-1 levels were quantified by ELISA (e; *p<0.0001, Mann-Whitney U test). Data are expressed as mean±SEM, n=6-18 mice/group from 2-3 independent experiments; *p<0.05 WT IL-33 treated mice (solid circles/solid bars) in comparison to PBS treated mice (solid squares/open bars), ^{#}p<0.05 Kit^{W-sh/W-sh} IL-33 treated mice (open circles/slashed bars) in comparison to PBS treated mice (open squares/spotted bars).

FIGS. 13a-13h: IL-33 protein levels were determined in ovalbumin (solid bars) and sham (open bars) sensitized and challenged mice in lung tissue homogenate supernatants, 1.5-24 hours after the final challenge, by ELISA (a; 1.5 h, 3 h, 6 h:*p=0.0002; 24 h:*p<0.0001, Mann-Whitney U test, n=14-17 mice/group from 1-2 independent experiments). (b-g; IL-33 immunoreactivity in the airway epithelium is restricted to cell nuclei with a clear distinction between intensively stained basal cell nuclei and less intensive staining of columnar epithelial cells. Scattered non-stained epithelial cells (exemplified in inset in b and upper c) were frequently observed. The subepithelial tissue harboured IL-33⁺endothelial cells (arrowheads in c) with a strict nuclear staining together with scattered cells displaying a cytoplasmatic immunoreactivity with a granulated appearance (asterisk in c and d), a phenomenon that was absent in control sections where the primary antibody has been omitted (e). Double immunofluorescence revealed IL-33⁺granules present in tryptase-positive mast cells (f). As indicated in d, generally only a some fraction of the granules in mast cells displayed IL-33 staining (g). Scale bars: c 40 μm, d-g 12 μm. Since immunostaining of asthmatic lung sections implicated mast cells as a potential source of IL-33, mouse BMMCs were stimulated accordingly and samples were analyzed for IL-33 mRNA expression. While IL-33 was not detected in IgE receptor activated BMMCs, a time related increase in mRNA expression in IgE receptor activated+IL-33 stimulated cells was detected. This increase was seen at 90 mins and 4 h but had disappeared by 24 h suggesting de novo synthesis of IL-33 in mast cells (h).

FIGS. 14a-14e: (a) IL-33 induces a potent response (filled columns) when compared to IgER CL (hatched columns), LPS (checkered columns), and Pam3CSK4 stimulation (shaded columns) in the production of IL-6 from BMMC at 24 h. (b) T1/ST2 antibody inhibits the production of IL-6 and IL-13. BMMC were pre-incubated with T1/ST2 antibody (1-10 ug/ml) for 30 minutes prior to addition of IL-33 (0.01 ng/ml). (c) IL-33 alone (solid columns) or in combination with IgER CL (checkered columns) did not enhance histamine production. (d) The expression of mMCP-1 in BMMC was upregulated upon stimulation with 10 ng/ml of IL-33 (solid columns) in a time course fashion beginning at 1.5 h and reaching a maximal induction at 4 h. (e) IL-5 levels and IL-13 levels in culture supernatants of HMC were measured by ELISA 24 h after exposure to IL-33, with (checkered columns) or without IgER CL (solid columns). Supernatants from HMC stimulated by IgER CL for 24 h without IL-33 are shown as hatched columns. HMC results are representative of two independent experiments obtained with different batches of cells. BMMC results are representative of two independent experiments obtained with different batches of cells. All data are expressed as means±SEM.

FIG. 15: IL-33 induces secretion of various mediators by BMMC. BMMCs were stimulated with IL-33 (10 ng/ml), IgER CL, or in combination for 24 hours. Data are expressed as means±SEM.

FIGS. 16a-16b: IL-33 induced expression of Th2 cytokines One (hatched bars), two (striped bars) or three (solid bars) doses of IL-33 or PBS (open bars) was delivered intranasally to naïve mice, and lung inflammation was assessed at 1, 2 and 3 days post-administration. Expression of the Th2 cytokines IL-5 (a; *p<0.0001) and IL-13 (b; *p<0.0001) was assessed by Taqman. Data are expressed as mean relative expression compared to GAPDH±SEM, n=8-17 mice/group, *p<0.05 in comparison to PBS treated mice, Mann-Whitney U test.

FIGS. 17a-17c: AHR induced by three doses of IL-33 was not dependent on CD4 cells. Mice were treated with a depleting antibody against CD4 prior to IL-33 administration, and AHR was assessed 3 days later. AHR was assessed by measuring changes in tissue resistance (G; a), tissue elastance (H; b), and Newtonian resistance of the airways (Rₙ; c), in response to increasing concentrations of methacholine. Data are expressed as mean±SEM, n=6-18 mice/group; *p<0.05 IL-33+Ig treated mice (solid circles) in comparison to PBS+Ig treated mice (solid squares); ^{#}p<0.05 IL-33+anti-CD4 treated mice (open circles) in comparison to PBS+anti-CD4 treated mice (open squares), two-way ANOVA.

FIGS. 18a-18e: AHR induced by one dose of IL-33 was not dependent on CD4 cells. Mice were treated with a depleting antibody against CD4 prior to IL-33 administration, and AHR was assessed 1 day later. AHR was assessed by measuring changes in resistance (a), elastance (b), tissue resistance (G; c), tissue elastance (H; d), and Newtonian resistance of the airways (e Rₙ), in response to increasing concentrations of methacholine. Data are expressed as mean±SEM, n=6-18 mice/group; *p<0.05 IL-33+Ig treated mice (solid triangles) in comparison to PBS+Ig treated mice (solid squares); ^{#}p<0.05 IL-33+anti-CD4 treated mice (open triangles) in comparison to PBS+anti-CD4 treated mice (open squares), two-way ANOVA.

FIGS. 19a-19e: IL-33 induced inflammation was not dependent on CD4 cells. Mice were treated with a depleting antibody against CD4 prior to IL-33 administration, and inflammation was assessed 1 day after the final dose of IL-33. Leukocytes were isolated from the airway lumen and phenotyped (a; Total:*p=0.0087, ^{#}p=0.0012; Eo:*p=0.026, ^{#}p=0.0037; Mac:*p=0.026, ^{#}p=0.0037; Lymph:*p=0.0260; Neut:*p=0.0152, ^{#}p=0.0003). Mucin-related gene (b; MUC5ac:*p=0.0009, ^{#}p=0.0001; Gob-5:*p=0.0009, p=0.0001) and IL-13 (c; *p=0.0009, p=0.0001) expression was assessed by Taqman. IL-13 protein levels were determined in BAL supernatant (d; *p=0.0009, ^{#}p=0.0002) and lung tissue homogenate supernatant (e; *p=0.0011, p=0.0001). Data are expressed as mean±SEM, n=6-18 mice/group; *p<0.05 IL-33+Ig treated mice (solid bars) in comparison to PBS+Ig treated mice (open bars); ^{#}p<0.05 IL-33+anti-CD4 treated mice (slashed bars) in comparison to PBS+anti-CD4 treated mice (grey bars), Mann-Whitney U test.

FIGS. 20a-20c. AHR induced by 3 doses of IL-33 was greatly reduced in mast cell deficient mice. WT and Kit^{W-sh/W-sh} mice were treated with 3 intranasal doses of IL-33, and AHR was assessed 1 day after the final dose. AHR was assessed by measuring changes in tissue resistance (G; a), tissue elastance (H; b), and Newtonian resistance of the airways (c; Rₙ) in response to increasing concentrations of methacholine. Data are expressed as mean±SEM, n=16-22 mice/group from 3 independent experiments; *p<0.05 WT IL-33 treated mice (solid circles) in comparison to PBS treated mice (solid squares), ^{#}p<0.05 Kit^{W-sh/W-sh} IL-33 treated mice (open circles) in comparison to PBS treated mice (open squares), two-way ANOVA.

FIGS. 21a-21e: Mast cell deficient mice were protected from AHR induced by one dose of IL-33. WT (IL-33:solid triangles; PBS:solid squares) and Kit^{W-sh/W-sh} (IL-33:open triangles; PBS:open squares) mice were treated with one intranasal dose of IL-33, and AHR was assessed 1 day later. AHR was assessed by measuring changes in resistance (a), elastance (b), tissue resistance (G; c), tissue elastance (H; d), and Newtonian resistance of the airways (Rₙ; e), in response to increasing concentrations of methacholine. Data are expressed as mean±SEM, n=16-22 mice/group from 3 independent experiments; *p<0.05 IL-33 treated mice in comparison to PBS treated mice, Mann-Whitney U test.

FIGS. 22a-22f: IL-33 induced inflammation in mast cell deficient mice. WT and Kit^{W-sh/W-sh} mice were treated with 3 intranasal doses of IL-33, and inflammation was assessed 1 day after the final dose. Leukocytes were isolated from the airway lumen (a; Total:*p=0.0003, ^{#}p=0.002; Eo:*p=0.0002, ^{#}p=0.0007; Mac:*p=0.0112, ^{#}p=0.0481; Lymph:*p=0.0074, ^{#}p=0.0278; Neut:*p=0.0137, ^{#}p=0.002) and lung tissue (b; Total:*p=0.0003, ^{#}p=0.002; Eo:*p=0.0002, ^{#}p=0.0007; Mac:*p=0.0112, ^{#}p=0.0481; Lymph:*p=0.0074, ^{#}p=0.0278; Neut:*p=0.0137, ^{#}p=0.0020). The CD4 response in lung tissue (c; CD4:*p=0.0016, ^{#}p=0.0051; CD4/T1/ST2:*p=0.0016, ^{#}p=0.0051) was measured as previously described. Mucin-related (d; MUC5ac:*p=0.0061, ^{#}p=0.0167; Gob-5:*p=0.0061, ^{#}p=0.0167) and IL-13 (e; *p=0.0061, ^{#}p=0.0167) gene expression was assessed by taqman analysis. IL-13 protein levels (f; *p=0.0006, ^{#}p=0.0006) were determined in lung homogenate supernatant by ELISA. Data are expressed as mean±SEM, n=4-22 mice/group from 1-3 independent experiments; *p<0.05 WT IL-33 treated mice (solid bars) in comparison to PBS treated mice (open bars), ^{#}p<0.05 Kit^{W-sh/W-sh} IL-33 treated mice (slashed bars) in comparison to PBS treated mice (spotted bars).

FIGS. 23a-23d: Allergen challenge induces IL-13 and IL-33 production in vivo. BALB/c mice were immunized, (Ovalbumin, OVA), challenged via the airways with OVA and the lung analyzed at various times for IL-13 protein (a). Expression of IL-33 in human asthmatic lung biopsies was also examined. The most intense staining was present in the nuclei of structural cells, foremost in epithelial cells (b) and also in endothelial cells (c and d). The epithelial staining was consistent between biopsies and mainly localized to basal cells although consistent but weak nuclei staining was observed within the columnar epithelial cells (i.e. ciliated cells and goblet cells). Furthermore, endothelial cells of bronchial blood vessels frequently displayed IL-33 immunoreactivity (c and d).

### DETAILED DESCRIPTION

The invention encompasses IL-33 specific binding polypeptides and compositions and compositions comprising IL-33 specific polypeptides. These IL-33 specific binding polypeptides may comprise antibodies, monomer domain polypeptides, or multimer domain polypeptides. The IL-33 specific binding polypeptides may be used to treat disorders including autoimmune disorders and inflammatory disorders, e.g., asthma.

### Disorders/Diseases

IL-33 specific binding polypeptides can be used to treat any inflammatory disorder such as chronic inflammatory disorders. Chronic inflammatory disorders include rheumatoid arthritis, psoriasis, allergy, asthma, COPD, inflammatory bowel diseases (e.g., Crohn's disease and ulcerative colitis), and autoimmune thyroiditis (e.g., Graves' disease and Hashimoto's thyroiditis). A review of disorders is provided in Girard and Springer, (1995) Immunology Today 16(9): 449-457.

IL-33 specific binding polypeptides can also be used to treat disorders characterized by extralymphoid sites of chronic inflammation. For instance, IL-33 inhibitors may be useful for the treatment or prevention of diabetes mellitus. An IL-33 specific binding polypeptide may be used for the treatment or prevention of graft rejection.

Inflammatory diseases or disorders also encompass any disorder or pathological condition where the pathology results, in whole or in part, from, e.g., a change in number, change in rate of migration, or change in activation, of cells of the immune system. Cells of the immune system include, e.g., T cells, B cells, monocytes or macrophages, antigen presenting cells (APCs), dendritic cells, microglia, NK cells, NKT cells, neutrophils, eosinophils, mast cells, or any other cell specifically associated with the immunology, for example, cytokine-producing endothelial or epithelial cells.

### Subject

In methods of treating a disease or disorder, a subject or patient is administered an IL-33 specific binding polypeptide. Subjects or patients may be animals, e.g., a mammal including a non-primate (e.g., a cow, pig, horse, cat, dog, rat, and mouse) or a primate (e.g., a monkey, such as a cynomolgous monkey, chimpanzee, and a human). The subjects or patients may be mammals, e.g., a human, with a disease or disorder. The subjects or patients may be farm animals (e.g., a horse, pig, or cow) or pets (e.g., a dog or cat) with a disorder. The subject may be a mammal (e.g., an immunocompromised or immunosuppressed mammal), at risk of developing a disorder or have one or more symptoms of a disorder.

IL-33 Binding Polypeptide

Subjects may be treated by administering a binding polypeptide. A binding polypeptide may be any molecule polypeptide, small molecule, macromolecule, antibody, a fragment or analogue thereof, monomer/multimer domain polypeptide, or soluble receptor, capable of binding to a IL-33 or IL-33R. A binding polypeptide also may refer to a complex of molecules, e.g., a non-covalent complex, to an ionized molecule, and to a covalently or non-covalently modified molecule, e.g., modified by phosphorylation, acylation, cross-linking, cyclization, or limited cleavage, which is capable of binding to IL-33 or IL-33R. A binding polypeptide may also refer to a molecule in combination with a stabilizer, excipient, salt, buffer, solvent, or additive, capable of binding to a target.

### Antibodies

The IL-33 specific binding polypeptide may be an IL-33 specific antibody. A composition comprising an IL-33 specific binding polypeptide may comprise an IL-33 specific antibody. A composition comprising an IL-33 specific antibody may comprise the IL-33 specific antibody substantially free of cellular material or heterologous contaminating proteins, e.g., the IL-33 specific antibody may be in an isolated form. For example, such compositions may have less than about 30%, 20%, 10%, 5% (by dry weight) of chemical precursors, cellular components, or heterologous contaminating proteins other than the IL-33 specific antibody. Such a composition may be combined with another therapeutic agent.

Antibodies include immunoglobulin molecules and immunologically active portions of immunoglobulin molecules, i.e., molecules that contain an antigen binding site which specifically binds (immunoreacts with) IL-33. Examples of immunologically active portions of immunoglobulin molecules include F(ab) and F(ab′)₂ fragments which can be generated by treating the antibody with an enzyme such as pepsin. The invention encompasses polyclonal and monoclonal antibodies that bind IL-33. Monoclonal antibody or monoclonal antibody composition encompasses a population of antibody molecules that contain antibodies capable of immunoreacting with a particular epitope of IL-33. A monoclonal antibody composition thus may display a single binding affinity for a particular IL-33 protein with which it immunoreacts.

IL-33 antibodies, either polyclonal or monoclonal, may be capable of selectively binding, or selectively bind to an epitope-containing a polypeptide comprising a contiguous span of at least 6 amino acids, preferably at least 8 to 10 amino acids, more preferably at least 12, 15, 20, 25, 30, 40, 50, 100, or more than 100 amino acids in a sequence of an IL-33 polypeptide or a mutated IL-33 polypeptide.

Polyclonal anti-IL-33 antibodies can be prepared by immunizing a suitable subject with an IL-33 immunogen. The anti-IL-33 antibody titer in the immunized subject can be monitored over time by standard techniques, such as with an enzyme linked immunosorbent assay (ELISA) using immobilized IL-33. If desired, the antibody molecules directed against IL-33 can be isolated from the mammal (e.g., from the blood) and further purified by well known techniques, such as protein A chromatography to obtain the IgG fraction. At an appropriate time after immunization, e.g., when the anti-IL-33 antibody titers are highest, antibody-producing cells can be obtained from the subject and used to prepare monoclonal antibodies by standard techniques, such as those described in the following references: the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a IL-33 immunogen as described above, and the culture supernatants of the resulting hybridoma cells are screened to identify a hybridoma producing a monoclonal antibody that binds IL-33.

Any of the many well known protocols used for fusing lymphocytes and immortalized cell lines can be applied for the purpose of generating an anti-IL-33 monoclonal antibody (see, e.g., G. Galfre et al. (1977) Nature 266:55052; Gefter et al. Somatic Cell Genet., cited supra; Lerner, Yale J. Biol. Med, cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from American Type Culture Collection (ATCC). Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind IL-33, e.g., using a standard ELISA assay.

Alternative to preparing monoclonal antibody-secreting hybridomas, a monoclonal anti-IL-33 antibody can be identified and isolated by screening a recombinant combinatorial immunoglobulin library (e.g., an antibody phage display library) with IL-33 to thereby isolate immunoglobulin library members that bind IL-33. Kits for generating and screening phage display libraries are commercially available (e.g., the Pharmacia Recombinant Phage Antibody System, Catalog No. 27-9400-01; and the Stratagene SurfzAP.™. Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc. Acid Res. 19:4133-4137; Barbas et al. (1991) PNAS 88:7978-7982; and McCafferty et al. Nature (1990) 348:552-554.

Additionally, recombinant anti-IL-33 antibodies, such as chimeric and humanized monoclonal antibodies, comprising both human and non-human portions, which can be made using standard recombinant DNA techniques, are within the scope of the invention.

Such chimeric and humanized monoclonal antibodies can be produced by recombinant DNA techniques known in the art, for example using methods described in Robinson et al. International Application No. PCT/US86/02269; Akira, et al. European Patent Application 184, 187; Taniguchi, M., European Patent Application 171, 496; Morrison et al. European Patent Application 173, 494; Neuberger et al. PCT International Publication No. WO 86/01533; Cabilly et al. U.S. Pat. No. 4,816,567; Cabilly et al. European Patent Application 125, 023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

Muteins and variants of antibodies and soluble receptors are contemplated, e.g., pegylation or mutagenesis to remove or replace deamidating Asn residues.

An anti-IL-33 antibody (e.g., monoclonal antibody) can be used to isolate IL-33 by standard techniques, such as affinity chromatography or immunoprecipitation. An anti-IL-33 antibody can facilitate the purification of natural IL-33 from cells and of recombinantly produced IL-33 expressed in host cells. Moreover, an anti-IL-33 antibody can be used to detect IL-33 protein (e.g., in a cellular lysate or cell supernatant) in order to evaluate the abundance and pattern of expression of the IL-33 protein. Anti-IL-33 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

### Monomer/Multimer Domain Polypeptides

The IL-33 specific binding polypeptide may be an IL-33 specific monomer/multimer domain polypeptide. Composition may comprise an IL-33 specific monomer/multimer domain polypeptide. A composition comprising an IL-33 monomer/multimer domain polypeptide may comprise the IL-33 specific antibody substantially free of cellular material or heterologous contaminating proteins, e.g., the IL-33 specific antibody may be in an isolated form. For examples, such compositions may have less than about 30%, 20%, 10%, 5% (by dry weight) of chemical precursors, cellular components, or heterologous contaminating proteins other than the IL-33 specific antibody. Such a composition may be combined with another therapeutic agent.

IL-33 specific monomer/multimer domain polypeptides may comprise a monomer domain that binds to IL-33, which monomer is of any size. A monomer domain may have about 25 to about 500, about 30 to about 200, about 30 to about 100, about 35 to about 50, about 35 to about 100, about 90 to about 200, about 30 to about 250, about 30 to about 60, about 9 to about 150, about 100 to about 150, about 25 to about 50, or about 30 to about 150 amino acids. Monomer domains may comprise, e.g., from about 30 to about 200 amino acid residues; from about 25 to about 180 amino acids; from about 40 to about 150 amino acids; from about 50 to about 130 amino acids; or from about 75 to about 125 amino acids.

Publications describing monomer domain polypeptides and mosaic polypeptides and references cited within include the following: Hegyi, H. and Bork, P., On the classification and evolution of protein modules, (1997) J. Protein Chem., 16(5):545-551; Baron et al., Protein modules (1991) Trends Biochem. Sci. 16(1):13-7; Ponting et al., Evolution of domain families, (2000), Adv. Protein Chem., 54:185-244; Doolittle, The multiplicity of domains in proteins, (1995) Annu Rev. Biochem 64:287-314; Doolitte and Bork, Evolutionarily mobile modules in proteins (1993) Scientific American, 269 (4):50-6; and Bork, Shuffled domains in extracellular proteins (1991), FEBS letters 286(1-2):47-54. Monomer domains of the present invention also include those domains found in Pfam database and the SMART database. See Schultz, et al., SMART: a web-based tool for the study of genetically mobile domains, (2000) Nucleic Acid Res. 28(1):231-34. Illustrative monomer domains include, e.g., an EGF-like domain, a Kringle-domain, a fibronectin type I domain, a fibronectin type II domain, a fibronectin type III domain, a PAN domain, a G1a domain, a SRCR domain, a Kunitz/Bovine pancreatic trypsin Inhibitor domain, a Kaza1-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an Immunoglobulin-like domain, a C-type lectin domain, a MAM domain, a von Willebrand factor type A domain, a Somatomedin B domain, a WAP-type four disulfide core domain, a F5/8 type C domain, a Hemopexin domain, an SH2 domain, an SH3 domain, a Laminin-type EGF-like domain, a C2 domain, and other such domains known to those of ordinary skill in the art, as well as derivatives and/or variants thereof.

Monomer domain polypeptides may include (1) β sandwich domains; (2) β-barrel domains; or (3) cysteine-rich domains comprising disulfide bonds. Cysteine-rich domains employed in the practice of the present invention typically do not form an α helix, a β sheet, or a β-barrel structure. Disulfide bonds may promote folding of the domain into a three-dimensional structure. Cysteine-rich domains may have at least two disulfide bonds, more typically at least three disulfide bonds.

Domains of the included in the monomer or multimer domain polypeptides can have any number of characteristics. For example, the domains may have low or no immunogenicity in an animal (e.g., a human). Domains may have a small size. The domains may be small enough to penetrate skin or other tissues. Domains may have a range of in vivo half-lives or stabilities.

Characteristics of a monomer domain may include the ability to fold independently and the ability to form a stable structure. Thus, the structure of the monomer domain is often conserved, although the polynucleotide sequence encoding the monomer need not be conserved. For example, the A-domain structure is conserved among the members of the A-domain family, while the A-domain nucleic acid sequence is not. Thus, for example, a monomer domain may be classified as an A-domain by its cysteine residues and its affinity for calcium, not necessarily by its nucleic acid sequence.

As described herein, monomer domains are selected for the ability to bind to IL-33, which may or may not be the target of a homologous naturally occurring domain may bind.

The monomer domain that specifically binds IL-33 may be a human A-domain Proteins containing A-domains include, e.g., complement components (e.g., C6, C7, C8, C9, and Factor I), serine proteases (e.g., enteropeptidase, matriptase, and corin), transmembrane proteins (e.g., ST7, LRP3, LRP5 and LRP6) and endocytic receptors (e.g., Sortilin-related receptor, LDL-receptor, VLDLR, LRP1, LRP2, and ApoER2). A domains and A domain variants can be readily employed in the practice of the present invention as monomer domains and variants thereof. Further description of A domains can be found in the following publications and references cited therein: Howell and Hertz, The LDL receptor gene family: signaling functions during development, (2001) Current Opinion in Neurobiology 11:74-81; Herz (2001), supra; Krieger, The “best” of cholesterols, the “worst” of cholesterols: A tale of two receptors, (1998) PNAS 95: 4077-4080; Goldstein and Brown, The Cholesterol Quartet, (2001) Science, 292: 1310-1312; and, Moestrup and Verroust, Megalin- and Cubilin-Mediated Endocytosis of Protein-Bound Vitamins, Lipids, and Hormones in Polarized Epithelia, (2001) Ann. Rev. Nutr. 21:407-28.

The monomer that specifically binds IL-33 may be derived from a C2 domain. C2 monomer domains are polypeptides containing a compact β-sandwich composed of two, four-stranded β-sheets, where loops at the “top” of the domain and loops at the “bottom” of the domain connect the eight β-strands. C2 monomer domains may be divided into two subclasses, namely C2 monomer domains with topology I (synaptotagmin-like topology) and topology II (cytosolic phospholipase A2-like topology), respectively. C2 monomer domains with topology I contains three loops at the “top” of the molecule (all of which are Ca²⁺ binding loops), whereas C2 monomer domains with topology II contain four loops at the “top” of the molecule (out of which only three are Ca²⁺ binding loops). The structure of C2 monomer domains have been reviewed by Rizo and Sudhof, J. Biol. Chem. 273; 15879-15882 (1998) and by Cho, J. Biol. Chem. 276; 32407-32410 (2001). The terms “loop region 1”, “loop region 2” and “loop region 3” refer to the Ca²⁺ binding loop regions located at the “top” of the molecule. This nomenclature, which is used to distinguish the three Ca²⁺ binding loops located at the “top” of the molecule from the non-Ca²⁺ binding loops (mainly located at the “bottom” of the molecule) is widely used and recognized in the literature. See Rizo and Sudhof, J. Biol. Chem. 273; 15879-15882 (1998). Loop regions 1, 2, and 3 represent target binding regions and thus can be varied to modulate binding specificity and affinity. The remaining portions of the C2 domain can be maintained without alteration if desired.

Other examples of monomer domains that may specifically bind IL-33 or may be altered to specifically bind IL-33 can be found in the protein Cubilin, which contains EGF-type repeats and CUB domains. The CUB domains are involved in ligand binding, e.g., some ligands include intrinsic factor (IF)-vitamin B12, receptor associated protein (RAP), Apo A-I, Transferrin, Albumin, Ig light chains and calcium. See, Moestrup and Verroust, supra.

Further examples of monomer domains that may be specific for or may be altered to be specific for IL-33 those described in: Yamazaki et al., Elements of Neural Adhesion Molecules and a Yeast Vacuolar Protein Sorting Receptor are Present in a Novel Mammalian Low Density Lipoprotein Receptor Family Member, (1996) Journal of Biological Chemistry 271(40) 24761-24768; Nakayama et al., Identification of High-Molecular-Weight Proteins with Multiple EGF-like Motifs by Motif-Trap Screening, (1998) Genomics 51:27-34; Liu et al, Genomic Organization of New Candidate Tumor Suppressor Gene, LRP1B, (2000) Genomics 69:271-274; Liu et al., The Putative Tumor Suppressor LRP1B, a Novel Member of the Low Density Lipoprotein (LDL) Receptor Family, Exhibits Both Overlapping and Distinct Properties with the LDL Receptor-related Protein, (2001) Journal of Biological Chemistry 276(31):28889-28896; Ishii et al, cDNA of a New Low-Density Lipoprotein Receptor-Related Protein and Mapping of its Gene (LRP3) to Chromosome Bands 19q12-q13.2, (1998) Genomics 51:132-135; Orlando et al, Identification of the second cluster of ligand-binding repeats in megalin as a site for receptor-ligand interactions, (1997) PNAS USA 94:2368-2373; Jeon and Shipley, Vesicle-reconstituted Low Density Lipoprotein Receptor, (2000) Journal of Biological Chemistry 275(39):30458-30464; Simmons et al., Human Low Density Lipoprotein Receptor Fragment, (1997) Journal of Biological Chemistry 272(41):25531-25536; Fass et al., Molecular Basis of familial hypercholesterolaemia from structure of LDL receptor module, (1997) Nature 388:691-93; Daly et al., Three-dimensional structure of a cysteine-rich repeat from the low-density lipoprotein receptor, (1995) PNAS USA 92:6334-6338; North and Blacklow, Structural Independence of Ligand-Binding Modules Five and Six of the LDL Receptor, (1999) Biochemistry 38:3926-3935; North and Blacklow, Solution Structure of the Sixth LDL-A module of the LDL Receptor, (2000) Biochemistry 39:25640-2571; North and Blacklow, Evidence that Familial Hypercholesterolemia Mutations of the LDL Receptor Cause Limited Local Misfolding in an LDL-A Module Pair, (2000) Biochemistry 39:13127-13135; Beglova et al., Backbone Dynamics of a Module Pair from the Ligand-Binding Domain of the LDL Receptor, (2001) Biochemistry 40:2808-2815; Bieri et al., Folding, Calcium binding, and Structural Characterization of a Concatemer of the First and Second Ligand-Binding Modules of the Low-Density Lipoprotein Receptor, (1998) Biochemistry 37:10994-11002; Jeon et al., Implications for familial hypercholesterolemia from the structure of the LDL receptor YWTD-EGF domain pair, (2001) Nature Structural Biology 8(6):499-504; Kurniawan et al., NMR structure of a concatemer of the first and second ligand-binding modules of the human low-density lipoprotein receptor, (2000) Protein Science 9: 1282-1293; Esser et al., Mutational Analysis of the Ligand Binding Domain of the Low Density Lipoprotein Receptor, (1988) Journal of Biological Chemistry 263(26): 13282-13290; Russell et al., Different Combinations of Cysteine-rich Repeats Mediate Binding of Low Density Lipoprotein Receptor to Two Different Proteins, (1989) Journal of Biological Chemistry 264(36):21682-21688; Davis et al., Acid-dependent ligand dissociation and recycling of LDL receptor mediated by growth factor homology region, (1987) Nature 326:760-765; Rong et al., Conversion of a human low-density lipoprotein receptor ligand-binding repeat to a virus receptor: Identification of residues important for ligand specificity, (1998) PNAS USA 95:8467-8472; Agnello et al., Hepatitis C virus and other Flaviviridae viruses enter cells via low density lipoprotein receptor; (1999) PNAS 96(22):12766-12771; Esser and Russell, Transport-deficient Mutations in the Low Density lipoprotein receptor, (1988) Journal of Biological Chemistry 263(26):13276-13281; Davis et al., The Low Density Lipoprotein Receptor, (1987) Journal of Biological Chemistry 262(9):4075-4082; and, Peacock et al., Human Low Density Lipoprotein Receptor Expressed in Xenopus Oocytes, (1988) Journal of Biological Chemistry 263(16):7838-7845.

Further examples of monomer domains (e.g., VLDLR, ApoER2 and LRP1 proteins and their monomer domains) that may be specific for or may be altered to be specific for IL-33 those described in: Savonen et al., The Carboxyl-terminal Domain of Receptor-associated Protein Facilitates Proper Folding and Trafficking of the Very Low Density Lipoprotein Receptor by Interaction with the Three Amino-terminal Ligand-binding Repeats of the Receptor, (1999) Journal of Biological Chemistry 274(36):25877-25882; Hewat et al., The cellular receptor to human rhinovirus 2 binds around the 5-fold axis and not in the canyon: a structural view, (2000) EMBO Journal 19(23):6317-6325; Okun et al., VLDL Receptor Fragments of Different Lengths Bind to Human Rhinovirus HR V2 with Different Stoichiometry, (2001) Journal of Biological Chemistry 276(2):1057-1062; Rettenberger et al., Ligand Binding Properties of the Very Low Density Lipoprotein Receptor, (1999) Journal of Biological Chemistry 274(13):8973-8980; Mikhailenko et al., Functional Domains of the very low density lipoprotein receptor: molecular analysis of ligand binding and acid-dependent ligand dissociation mechanisms, (1999) Journal of Cell Science 112:3269-3281; Brandes et al., Alternative Splicing in the Ligand Binding Domain of Mouse ApoE Receptor-2 Produces Receptor Variants Binding Reelin but not alpa2-macroglobulin, (2001) Journal of Biological Chemistry 276(25):22160-22169; Kim et al., Exon/Intron Organization, Chromosome Localization, Alternative Splicing, and Transcription Units of the Human Apolipoprotein E Receptor 2 Gene, (1997) Journal of Biological Chemistry 272(13):8498-8504; Obermoeller-McCormick et al., Dissection of receptor folding and ligand-binding property with functional minireceptors of LDL receptor-related protein, (2001) Journal of Cell Science 114(5):899-908; Horn et al., Molecular Analysis of Ligand Binding of the Second Cluster of Complement-type Repeats of the Low Density Lipoprotein Receptor-related Protein, (1997) Journal of Biological Chemistry 272(21):13608-13613; Neels et al., The Second and Fourth Cluster of Class A Cysteine-rich Repeats of the Low Density Lipoprotein Receptor-related Protein Share Ligand-binding Properties, (1999) Journal of Biological Chemistry 274(44):31305-31311; Obermoeller et al., Differential Functions of the Triplicated Repeats Suggest Two Independent Roles for the Receptor-Associated Protein as a Molecular Chaperone, (1997) Journal of Biological Chemistry 272(16):10761-10768; Andersen et al., Identification of the Minimal Functional Unit in the Low Density Lipoprotein Receptor-related Protein for Binding the Receptor-associated Protein (RAP), (2000) Journal of Biological Chemistry 275(28):21017-21024; Andersen et al., Specific Binding of alpha-Macroglobulin to Complement-Type Repeat CR4 of the Low-Density Lipoprotein Receptor-Related Protein, (2000) Biochemistry 39:10627-10633; Vash et al., Three Complement-Type Repeats of the Low-Density Lipoprotein Receptor-Related Protein Define a Common Binding Site for RAP, PAI-1, and Lactoferrin, (1998) Blood 92(9):3277-3285; Dolmer et al., NMR Solution Structure of Complement-like Repeat CR3 from the Low Density Lipoprotein Receptor-related Protein, (2000) Journal of Biological Chemistry 275(5):3264-3269; Huang et al., NMR Solution Structure of Complement-like Repeat CR8 from the Low Density Lipoprotein Receptor-related Protein, (1999) Journal of Biological Chemistry 274(20): 14130-14136; and Liu et al., Uptake of HIV-1 Tat protein mediated by low-density lipoprotein receptor-related protein disrupts the neuronal metabolic balance of the receptor ligands, (2000) Nature Medicine 6(12): 1380-1387.

Additional examples of monomer domains that may be specific for or may be altered to be specific for IL-33 those described in::FitzGerald et al, Pseudomonas Exotoxin-mediated Selection Yields Cells with Altered Expression of Low-Density Lipoprotein Receptor-related Protein, (1995) Journal of Cell Biology, 129: 1533-41; Willnow and Herz, Genetic deficiency in low density lipoprotein receptor-related protein confers cellular resistance to Pseudomonas exotoxin A, (1994) Journal of Cell Science, 107:719-726; Trommsdorf et al., Interaction of Cytosolic Adaptor Proteins with Neuronal Apolipoprotein E Receptors and the Amyloid Precursor Protein, (1998) Journal of Biological Chemistry, 273(5): 33556-33560; Stockinger et al., The Low Density Lipoprotein Receptor Gene Family, (1998) Journal of Biological Chemistry, 273(48): 32213-32221; Obermoeller et al., Ca+2 and Receptor-associated Protein are independently required for proper folding and disulfide bond formation of the low density lipoprotein receptor-related protein, (1998) Journal of Biological Chemistry, 273(35):22374-22381; Sato et al., 39-kDa receptor-associated protein (RAP) facilitates secretion and ligand binding of extracellular region of very-low-density-lipoprotein receptor: implications for a distinct pathway from low-density-lipoprotein receptor, (1999) Biochem. J., 341:377-383; Avromoglu et al, Functional Expression of the Chicken Low Density Lipoprotein Receptor-related Protein in a mutant Chinese Hamster Ovary Cell Line Restores Toxicity of Pseudomonas Exotoxin A and Degradation of alpha2-Macroglobulin, (1998) Journal of Biological Chemistry, 273(11) 6057-6065; Kingsley and Krieger, Receptor-mediated endocytosis of low density lipoprotein: Somatic cell mutants define multiple genes required for expression of surface-receptor activity, (1984) PNAS USA, 81:5454-5458; Li et al, Differential Functions of Members of the Low Density Lipoprotein Receptor Family Suggests by their distinct endocystosis rates, (2001) Journal of Biological Chemistry 276(21):18000-18006; and, Springer, An Extracellular beta-Propeller Module Predicted in Lipoprotein and Scavenger Receptors, Tyrosine Kinases, Epidermal Growth Factor Precursor, and Extracellular Matrix Components, (1998) J. Mol. Biol. 283:837-862.

Monomer domains that may be identified or selected for binding affinity for IL-33 may be formed into multimers. Any method resulting in selection of domains with specific binding for IL-33 can be used. For example, the methods can comprise providing a plurality of different nucleic acids, each nucleic acid encoding a monomer domain; translating the plurality of different nucleic acids, thereby providing a plurality of different monomer domains; screening the plurality of different monomer domains for binding IL-33; and, identifying members of the plurality of different monomer domains that bind IL-33.

If it is necessary to alter a naturally occurring monomer domain such that it is specific for IL-33, the monomer domain may from an ancestral domain, a chimeric domain, randomized domain, mutated domain, etc. For example, ancestral domains can be based on phylogenetic analysis. Chimeric domains can be domains in which one or more regions are replaced by corresponding regions from other domains of the same family. For example, chimeric domains can be constructed by combining loop sequences from multiple related domains of the same family to form novel domains with potentially lowered immunogenicity. Those of skill in the art will recognize the immunologic benefit of constructing modified binding domain monomers by combining loop regions from various related domains of the same family rather than creating random amino acid sequences. For example, by constructing variant domains by combining loop sequences or even multiple loop sequences that occur naturally in human LDL receptor class A-domains, the resulting domains may contain IL-33 binding properties but may not contain any immunogenic protein sequences because all of the exposed loops are of human origin. The combining of loop amino acid sequences in endogenous context can be applied to all or any monomer constructs. Thus methods for generating a library of chimeric monomer domains derived from human proteins, the method comprising: providing loop sequences corresponding to at least one loop from each of at least two different naturally occurring variants of a human protein, wherein the loop sequences are polynucleotide or polypeptide sequences; and covalently combining loop sequences to generate a library of at least two different chimeric sequences, wherein each chimeric sequence encodes a chimeric monomer domain having at least two loops. The chimeric domain may have at least four loops, or at least six loops. The IL-33 monomer/multimer domain polypeptide may have at least three types of loops that are identified by specific features, such as, potential for disulfide bonding, bridging between secondary protein structures, and molecular dynamics (i.e., flexibility). The three types of loop sequences may be a cysteine-defined loop sequence, a structure-defined loop sequence, and a B-factor-defined loop sequence.

Randomized domains are domains in which one or more regions are randomized. The randomization can be based on full randomization, or optionally, partial randomization based on natural distribution of sequence diversity. Such domains may produce IL-33—specific monomer/multimer domain polypeptides.

Non-natural monomer domains or altered monomer domains can be produced by a number of methods. Any method of mutagenesis, such as site-directed mutagenesis and random mutagenesis (e.g., chemical mutagenesis) can be used to produce variants. In some embodiments, error-prone PCR is employed to create variants. Additional methods include aligning a plurality of naturally occurring monomer domains by aligning conserved amino acids in the plurality of naturally occurring monomer domains; and, designing the non-naturally occurring monomer domain by maintaining the conserved amino acids and inserting, deleting or altering amino acids around the conserved amino acids to generate the non-naturally occurring monomer domain. In one embodiment, the conserved amino acids comprise cysteines. In another embodiment, the inserting step uses random amino acids, or optionally, the inserting step uses portions of the naturally occurring monomer domains. The portions could ideally encode loops from domains from the same family. Amino acids are inserted or exchanged using synthetic oligonucleotides, or by shuffling, or by restriction enzyme based recombination. Human chimeric domains of the present invention are useful for therapeutic applications where minimal immunogenicity is desired. The present invention provides methods for generating libraries of human chimeric domains. Human chimeric monomer domain libraries can be constructed by combining loop sequences from different variants of a human monomer domain, as described above. The loop sequences that are combined may be sequence-defined loops, structure-defined loops, B-factor-defined loops, or a combination of any two or more thereof.

Alternatively, a human chimeric domain library can be generated by modifying naturally occurring human monomer domains at the amino acid level, as compared to the loop level. To minimize the potential for immunogenicity, only those residues that naturally occur in protein sequences from the same family of human monomer domains are utilized to create the chimeric sequences. This can be achieved by providing a sequence alignment of at least two human monomer domains from the same family of monomer domains, identifying amino acid residues in corresponding positions in the human monomer domain sequences that differ between the human monomer domains, generating two or more human chimeric monomer domains, wherein each human chimeric monomer domain sequence consists of amino acid residues that correspond in type and position to residues from two or more human monomer domains from the same family of monomer domains. Libraries of human chimeric monomer domains can be employed to identify human chimeric monomer domains that bind to IL-33 by: screening the library of human chimeric monomer domains for binding IL-33, and identifying a human chimeric monomer domain that binds to IL-33. Suitable naturally occurring human monomer domain sequences employed in the initial sequence alignment step include those corresponding to any of the naturally occurring monomer domains described herein.

Human chimeric domain libraries of the present invention (whether generated by varying loops or single amino acid residues) can be prepared by methods known to those having ordinary skill in the art. Methods particularly suitable for generating these libraries are split-pool format and trinucleotide synthesis format as described in WO01/23401.

In accordance with the present invention, a library of human-like chimeric proteins is generated by: identifying human protein sequences from a database that correspond to proteins from the same family of proteins; aligning the human protein sequences from the same family of proteins to a reference protein sequence; identifying a set of subsequences derived from different human protein sequences of the same family, wherein each subsequence shares a region of identity with at least one other subsequence derived from a different naturally occurring human protein sequence; identifying a chimeric junction from a first, a second, and a third subsequence, wherein each subsequence is derived from a different naturally occurring human protein sequence, and wherein the chimeric junction comprises two consecutive amino acid residue positions in which the first amino acid position is occupied by an amino acid residue common to the first and second naturally occurring human protein sequence, but not the third naturally occurring human protein sequence, and the second amino acid position is occupied by an amino acid residue common to the second and third naturally occurring human protein sequence, and generating human-like chimeric protein molecules each corresponding in sequence to two or more subsequences from the set of subsequences, and each comprising one of more of the identified chimeric junctions.

Altered monomer domains can also be generated by providing a collection of synthetic oligonucleotides (e.g., overlapping oligonucleotides) encoding conserved, random, pseudorandom, or a defined sequence of peptide sequences that are then inserted by ligation into a predetermined site in a polynucleotide encoding a monomer domain. Similarly, the sequence diversity of one or more monomer domains can be expanded by mutating the monomer domain(s) with site-directed mutagenesis, random mutation, pseudorandom mutation, defined kernal mutation, codon-based mutation, and the like. The resultant nucleic acid molecules can be propagated in a host for cloning and amplification. In some embodiments, the nucleic acids are recombined.

A plurality of nucleic acids encoding monomer domains may be recombined and screened to produce a library of monomer domains that bind to IL-33. Selected monomer domain nucleic acids can also be back-crossed by recombining with polynucleotide sequences encoding neutral sequences (i.e., having insubstantial functional effect on binding), such as for example, by back-crossing with a wild-type or naturally-occurring sequence substantially identical to a selected sequence to produce native-like functional monomer domains. Generally, during back-crossing, subsequent selection is applied to retain the property, e.g., binding to the ligand.

Selection of monomer domains and/or immuno-domains that specifically bind IL-33 from a library of domains can be accomplished by a variety of procedures. For example, one method of identifying monomer domains and/or immuno-domains which have a desired property involves translating a plurality of nucleic acids, where each nucleic acid encodes a monomer domain and/or immuno-domain, screening the polypeptides encoded by the plurality of nucleic acids, and identifying those monomer domains and/or immuno-domains that bind to IL-33, thereby producing a selected monomer domain and/or immuno-domain. The monomer domains and/or immuno-domains expressed by each of the nucleic acids can be tested for their ability to bind to IL-33 by methods known in the art (i.e. panning, affinity chromatography, FACS analysis).

As mentioned above, selection of monomer domains and/or immuno-domains can be based on binding to IL-33. Other selections of monomer domains and/or immuno-domains can be based, e.g., on inhibiting or enhancing a specific function of IL-33. IL-33 activity can include, e.g., reduction in induction of chemokine secretion by mast cells. The selection can also include using high-throughput assays.

When a monomer domain and/or immuno-domain is selected based on its ability to bind IL-33, the selection basis can include selection based on a slow dissociation rate, which is usually predictive of high affinity. The valency of the ligand can also be varied to control the average binding affinity of selected monomer domains and/or immuno-domains. IL-33 can be bound to a surface or substrate at varying densities, such as by including a competitor compound, by dilution, or by other method known to those in the art.

Examples of other display systems include ribosome displays, a nucleotide-linked display (see, e.g., U.S. Pat. Nos. 6,281,344; 6,194,550, 6,207,446, 6,214,553, and 6,258,558), polysome display, cell surface displays and the like. The cell surface displays include a variety of cells, e.g., E. coli, yeast and/or mammalian cells. When a cell is used as a display, the nucleic acids, e.g., obtained by PCR amplification followed by digestion, are introduced into the cell and translated. Optionally, polypeptides encoding the monomer domains or the multimers of the present invention can be introduced, e.g., by injection, into the cell.

In some embodiments, variants are generated by recombining two or more different sequences from the same family of monomer domains and/or immuno-domains (e.g., the LDL receptor class A domain). Alternatively, two or more different monomer domains and/or immuno-domains from different families can be combined to form a multimer. In some embodiments, the multimers are formed from monomers or monomer variants of at least one of the following family classes: an EGF-like domain, a Kringle-domain, a fibronectin type I domain, a fibronectin type II domain, a fibronectin type III domain, a PAN domain, a G1a domain, a SRCR domain, a Kunitz/Bovine pancreatic trypsin Inhibitor domain, a Kaza1-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an Immunoglobulin-like domain, a C-type lectin domain, a MAM domain, a von Willebrand factor type A domain, a Somatomedin B domain, a WAP-type four disulfide core domain, a F5/8 type C domain, a Hemopexin domain, an SH2 domain, an SH3 domain, a Laminin-type EGF-like domain, a C2 domain and derivatives thereof. In another embodiment, the monomer domain and the different monomer domain can include one or more domains found in the Pfam database and/or the SMART database. Libraries produced by the methods above, one or more cell(s) comprising one or more members of the library, and one or more displays comprising one or more members of the library are also included in the present invention.

Multimers comprise at least two monomer domains and/or immuno-domains that bind IL-33. For example, multimers can comprise from 2 to about 10 monomer domains and/or immuno-domains, from 2 and about 8 monomer domains and/or immuno-domains, from about 3 and about 10 monomer domains and/or immuno-domains, about 7 monomer domains and/or immuno-domains, about 6 monomer domains and/or immuno-domains, about 5 monomer domains and/or immuno-domains, or about 4 monomer domains and/or immuno-domains. In some embodiments, the multimer comprises at least 3 monomer domains and/or immuno-domains. In view of the possible range of monomer domain sizes, the multimers of the invention may be, e.g., 100 kD, 90 kD, 80 kD, 70 kD, 60 kD, 50 kd, 40 kD, 30 kD, 25 kD, 20 kD, 15 kD, 10 kD or smaller or larger. Typically, the monomer domains have been pre-selected for binding to IL-33.

In some embodiments, each monomer domain specifically binds to IL-33. In some of these embodiments, each monomer binds to a different position (analogous to an epitope) on IL-33. Multiple monomer domains and/or immuno-domains that bind to IL-33 can result in an avidity effect resulting in improved avidity of the multimer for IL-33 compared to each individual monomer. In some embodiments, the multimer has an avidity of at least about 1.5, 2, 3, 4, 5, 10, 20, 50 or 100 times the avidity of a monomer domain alone.

Multimers can comprise a variety of combinations of monomer domains. For example, in a single multimer, the selected monomer domains can be the same or identical, optionally, different or non-identical. In addition, the selected monomer domains can comprise various different monomer domains from the same monomer domain family, or various monomer domains from different domain families, or optionally, a combination of both.

The selected monomer domains may be joined by a linker to form a single chain multimer. For example, a linker is positioned between each separate discrete monomer domain in a multimer. Typically, immuno-domains are also linked to each other or to monomer domains via a linker moiety. Linker moieties that can be readily employed to link immuno-domain variants together are the same as those described for multimers of monomer domain variants. Linker moieties suitable for joining immuno-domain variants to other domains into multimers are described herein.

Joining the selected monomer domains via a linker can be accomplished using a variety of techniques known in the art. For example, combinatorial assembly of polynucleotides encoding selected monomer domains can be achieved by restriction digestion and re-ligation, by PCR-based, self-priming overlap reactions, or other rembinant methods. The linker can be attached to a monomer before the monomer is identified for its ability to bind to IL-33 or after the monomer has been selected for the ability to bind IL-33.

The linker can be naturally-occurring, synthetic or a combination of both. For example, the synthetic linker can be a randomized linker, e.g., both in sequence and size. In one aspect, the randomized linker can comprise a fully randomized sequence, or optionally, the randomized linker can be based on natural linker sequences. The linker can comprise, e.g., a non-polypeptide moiety, a polynucleotide, a polypeptide or the like.

A linker can be rigid, or alternatively, flexible, or a combination of both. Linker flexibility can be a function of the composition of both the linker and the monomer domains that the linker interacts with. The linker joins two selected monomer domain, and maintains the monomer domains as separate discrete monomer domains. The linker can allow the separate discrete monomer domains to cooperate yet maintain separate properties such as multiple separate binding sites for IL-33 in a multime. In some cases, a disulfide bridge exists between two linked monomer domains or between a linker and a monomer domain. In some embodiments, the monmer domains and/or linkers comprise metal-binding centers.

Choosing a suitable linker for a specific case where two or more monomer domains (i.e. polypeptide chains) are to be connected may depend on a variety of parameters including, e.g. the nature of the monomer domains, and/or the stability of the peptide linker towards proteolysis and oxidation.

The linker polypeptide may predominantly include amino acid residues selected from the group consisting of Gly, Ser, Ala and Thr. For example, the peptide linker may contain at least 75% (calculated on the basis of the total number of residues present in the peptide linker), such as at least 80%, e.g. at least 85% or at least 90% of amino acid residues selected from the group consisting of Gly, Ser, Ala and Thr. The peptide linker may also consist of Gly, Ser, Ala and/or Thr residues only. The linker polypeptide should have a length, which is adequate to link two monomer domains in such a way that they assume the correct conformation relative to one another so that they retain the desired activity.

A suitable length for this purpose is a length of at least one and typically fewer than about 50 amino acid residues, such as 2-25 amino acid residues, 5-20 amino acid residues, 5-15 amino acid residues, 8-12 amino acid residues or 11 residues. Similarly, the polypeptide encoding a linker can range in size, e.g., from about 2 to about 15 amino acids, from about 3 to about 15, from about 4 to about 12, about 10, about 8, or about 6 amino acids. In methods and compositions involving nucleic acids, such as DNA, RNA, or combinations of both, the polynucleotide containing the linker sequence can be, e.g., between about 6 nucleotides and about 45 nucleotides, between about 9 nucleotides and about 45 nucleotides, between about 12 nucleotides and about 36 nucleotides, about 30 nucleotides, about 24 nucleotides, or about 18 nucleotides. Likewise, the amino acid residues selected for inclusion in the linker polypeptide should exhibit properties that do not interfere significantly with the activity or function of the polypeptide multimer. Thus, the peptide linker should on the whole not exhibit a charge which would be inconsistent with the activity or function of the polypeptide multimer, or interfere with internal folding, or form bonds or other interactions with amino acid residues in one or more of the monomer domains which would seriously impede the binding of the polypeptide multimer to IL-33.

The peptide linker may also be selected from a library where the amino acid residues in the peptide linker are randomized for a specific set of monomer domains in a particular polypeptide multimer. A flexible linker could be used to find suitable combinations of monomer domains, which is then optimized using this random library of variable linkers to obtain linkers with optimal length and geometry. The optimal linkers may contain the minimal number of amino acid residues of the right type that participate in the binding to the target and restrict the movement of the monomer domains relative to each other in the polypeptide multimer when not bound to IL-33

The use of naturally occurring as well as artificial peptide linkers to connect polypeptides into novel linked fusion polypeptides is well known in the literature (Hallewell et al. (1989), J. Biol. Chem. 264, 5260-5268; Alfthan et al. (1995), Protein Eng. 8, 725-731; Robinson & Sauer (1996), Biochemistry 35, 109-116; Khandekar et al. (1997), J. Biol. Chem. 272, 32190-32197; Fares et al. (1998), Endocrinology 139, 2459-2464; Smallshaw et al. (1999), Protein Eng. 12, 623-630; U.S. Pat. No. 5,856,456).

As mentioned above, it is generally preferred that the peptide linker possess at least some flexibility. Accordingly, in some embodiments, the peptide linker contains 1-25 glycine residues, 5-20 glycine residues, 5-15 glycine residues or 8-12 glycine residues. The peptide linker will typically contain at least 50% glycine residues, such as at least 75% glycine residues. In some embodiments of the invention, the peptide linker comprises glycine residues only.

In some cases it may be desirable or necessary to provide some rigidity into the peptide linker. This may be accomplished by including proline residues in the amino acid sequence of the peptide linker. Thus, in another embodiment of the invention, the peptide linker may comprise at least one proline residue in the amino acid sequence of the peptide linker. For example, the peptide linker has an amino acid sequence, wherein at least 25%, such as at least 50%, e.g. at least 75%, of the amino acid residues are proline residues. In one particular embodiment of the invention, the peptide linker comprises proline residues only.

In some embodiments of the invention, the peptide linker is modified in such a way that an amino acid residue comprising an attachment group for a non-polypeptide moiety is introduced. Examples of such amino acid residues may be a cysteine residue (to which the non-polypeptide moiety is then subsequently attached) or the amino acid sequence may include an in vivo N-glycosylation site (thereby attaching a sugar moiety (in vivo) to the peptide linker). An additional option is to genetically incorporate non-natural amino acids using evolved tRNAs and tRNA synthetases (see, e.g., U.S. patent application Publication Ser. No. 2003/0082575) into the monomer domains or linkers. For example, insertion of keto-tyrosine allows for site-specific coupling to expressed monomer domains or multimers.

Sometimes, the amino acid sequences of all peptide linkers present in the polypeptide multimer will be identical. Alternatively, the amino acid sequences of all peptide linkers present in the polypeptide multimer may be different.

Quite often, it will be desirable or necessary to attach only a few, typically only one, non-polypeptide moieties/moiety (such as mPEG, a sugar moiety or a non-polypeptide therapeutic agent) to the polypeptide multimer in order to achieve the desired effect, such as prolonged serum-half life. Evidently, in case of a polypeptide tri-mer, which will contain two peptide linkers, only one peptide linker may typically required to be modified, e.g. by introduction of a cysteine residue, whereas modification of the other peptide linker may typically not be necessary not. In this case all (both) peptide linkers of the polypeptide multimer (tri-mer) may be different.

Methods for evolving monomers or multimers can comprise, e.g., any or all of the following steps: providing a plurality of different nucleic acids, where each nucleic acid encoding a monomer domain; translating the plurality of different nucleic acids, which provides a plurality of different monomer domains; screening the plurality of different monomer domains for binding of IL-33; identifying members of the plurality of different monomer domains that bind IL-33, which provides selected monomer domains; joining the selected monomer domains with at least one linker to generate at least one multimer, wherein the at least one multimer comprises at least two of the selected monomer domains and the at least one linker; and, screening the at least one multimer for an improved affinity or avidity or altered specificity for IL-33 as compared to the selected monomer domains.

Variation can be introduced into either monomers or multimers. An example of improving monomers includes intra-domain recombination in which two or more (e.g., three, four, five, or more) portions of the monomer are amplified separately under conditions to introduce variation (for example by shuffling or other recombination method) in the resulting amplification products, thereby synthesizing a library of variants for different portions of the monomer. By locating the 5′ ends of the middle primers in a “middle” or ‘overlap’ sequence that both of the PCR fragments have in common, the resulting “left” side and “right” side libraries may be combined by overlap PCR to generate novel variants of the original pool of monomers. These new variants may then be screened for desired properties, e.g., panned against a target or screened for a functional effect. The “middle” primer(s) may be selected to correspond to any segment of the monomer, and will typically be based on the scaffold or one or more concensus amino acids within the monomer (e.g., cysteines such as those found in A domains).

Similarly, multimers may be created by introducing variation at the monomer level and then recombining monomer variant libraries. On a larger scale, multimers (single or pools) with desired properties may be recombined to form longer multimers. In some cases variation is introduced (typically synthetically) into the monomers or into the linkers to form libraries.

Additional variation can be introduced by inserting linkers of different length and composition between domains. This allows for the selection of optimal linkers between domains. In some embodiments, optimal length and composition of linkers will allow for optimal binding of domains. In some embodiments, the domains with a particular binding affinity(s) are linked via different linkers and optimal linkers are selected in a binding assay. For example, domains are selected for desired binding properties and then formed into a library comprising a variety of linkers. The library can then be screened to identify optimal linkers. Alternatively, multimer libraries can be formed where the effect of domain or linker on IL-33 binding is not known.

One method for identifying multimers can be accomplished by displaying the multimers. As with the monomer domains, the multimers are optionally expressed or displayed on a variety of display systems, e.g., phage display, ribosome display, polysome display, nucleotide-linked display (see, e.g., U.S. Pat. Nos. 6,281,344; 6,194,550, 6,207,446, 6,214,553, and 6,258,558) and/or cell surface display, as described above. Cell surface displays can include but are not limited to E. coli, yeast or mammalian cells. In addition, display libraries of multimers with multiple binding sites can be panned for avidity or affinity or altered specificity for IL-33.

In some embodiments, the monomer or multimer domain is linked to a molecule (e.g., a protein, nucleic acid, organic small molecule, etc.) useful as a pharmaceutical. Exemplary pharmaceutical proteins include, e.g., cytokines, antibodies, chemokines, growth factors, interleukins, cell-surface proteins, extracellular domains, cell surface receptors, cytotoxins, etc. Exemplary small molecule pharmaceuticals include small molecule toxins or therapeutic agents.

Multimers or monomer domains of the invention can be produced according to any methods known in the art. In some embodiments, E. coli comprising a pET-derived plasmid encoding the polypeptides are induced to express the protein. After harvesting the bacteria, they may be lysed and clarified by centrifugation. The polypeptides may be purified using Ni—NTA agarose elution and refolded by dialysis. Misfolded proteins may be neutralized by capping free sulfhydrils with iodoacetic acid. Q sepharose elution, butyl sepharose FT, SP sepharose elution, Q sepharose elution, and/or SP sepharose elution may be used to purify the polypeptides.

Monomer/multimer domain polypeptides are further described in U.S. Pat. Nos. 6,673,901, 7,153,661, and U.S. patent application publications 2005-0048512 and 2006-0223114.

IL-33 Specific Monomers Comprising a Fibronectin Domain/Scaffold

Monomers that specifically bind IL-3 may be based on fibronectin domains and comprise a fibronectin or fibronectin-like scaffold. These monomers act like antibody mimics that exhibit optimal folding, stability, and solubility, under conditions which normally lead to the loss of structure and function in antibodies.

IL-33 specific monomers comprising a fibronectin domain/scaffold comprise three fibronectin loops which are analogous to the complementarity determining regions (CDRs) of an antibody variable region. These loops may or may not be subjected to directed evolution designed to improve IL-33 binding. Such a directed evolution approach results in the production of antibody-like molecules with high affinities for antigens of interest. In addition, the fibronectin domains/scaffolds may be used to display defined exposed loops (for example, loops previously randomized and selected on the basis of antigen binding) in order to direct the evolution of molecules that bind to such introduced loops. A selection of this type may be carried out to identify recognition molecules for any individual CDR-like loop or, alternatively, for the recognition of two or all three CDR-like loops combined into a non-linear epitope.

A fibronectin domain/scaffold may be a fibronectin type III domain (Fn3). An Fn3 domain may be a domain having 7 or 8 beta strands which are distributed between two beta sheets, which themselves pack against each other to form the core of the protein, and further containing loops which connect the beta strands to each other and are solvent exposed. There are at least three such loops at each edge of the beta sheet sandwich, where the edge is the boundary of the protein perpendicular to the direction of the beta strands. Fn3 domains are commonly found in mammalian blood and structural proteins. Fn3 domains may be derived from any polypeptide; such polypeptides are known to include fibronectins, tenascin, intracellular cytoskeletal proteins, and prokaryotic enzymes (Bork and Doolittle, Proc. Natl. Acad. Sci. USA 89:8990, 1992; Bork et al., Nature Biotech. 15:553, 1997; Meinke et al., J. Bacteriol. 175:1910, 1993; Watanabe et al., J. Biol. Chem. 265:15659, 1990).

If the fibronectin domain is a Fn3 domain, the Fn3 domain may be a module of any one of ¹Fn3-⁹Fn3 and ¹¹Fn3-¹⁷Fn3, as well as related Fn3 modules from non-human animals and prokaryotes. In addition, Fn3 modules from other proteins with sequence homology to ¹⁰Fn3, such as tenascins and undulins, may also be used. Modules from different organisms and parent proteins may be most appropriate for different applications; for example, in designing an antibody mimic, it may be most desirable to generate that protein from a fibronectin or fibronectin-like molecule native to the organism for which a therapeutic or diagnostic molecule is intended.

A ¹⁰Fn3 module may have a sequence which exhibits at least 30% amino acid identity, or at least 50% amino acid identity, to the sequence encoding the structure of the ¹⁰Fn3 domain referred to as “1ttg” (ID=“1ttg” (one ttg)) available from the Protein Data Base. Sequence identity referred to in this definition is determined by the Homology program, available from Molecular Simulation (San Diego, Calif.). The ¹⁰Fn3 module may also be a polymer of a ¹⁰Fn3-related module, which may be an extension of the use of the monomer structure, whether or not the subunits of the polyprotein are identical or different in sequence. ¹⁰Fn3 modules typically comprise 94 amino acid residues. The overall fold of this domain is closely related to that of the smallest functional antibody fragment, the variable region of the heavy chain, which comprises the entire antigen recognition unit in camel and llama IgG). The major differences between camel and llama domains and the ¹⁰Fn3 domain are that (i) ¹⁰Fn3 has fewer beta strands (seven vs. nine) and (ii) the two beta sheets packed against each other are connected by a disulfide bridge in the camel and llama domains, but not in ¹⁰Fn3.

The three loops of ¹⁰Fn3 corresponding to the antigen-binding loops of the IgG heavy chain run about between amino acid residues 21-31, 51-56, and 76-88. The length of the first and the third loop, 11 and 12 residues, respectively, fall within the range of the corresponding antigen-recognition loops found in antibody heavy chains, that is, 10-12 and 3-25 residues, respectively. Accordingly, once randomized and selected for high antigen affinity, these two loops make contacts with IL-33 equivalent to the contacts of the corresponding loops in antibodies.

In contrast, the second loop of ¹⁰Fn3 is only 6 residues long, whereas the corresponding loop in antibody heavy chains ranges from 16-19 residues. To optimize antigen binding, therefore, the second loop of ¹⁰Fn3 may be extended by 10-13 residues (in addition to being randomized) to obtain the greatest possible flexibility and affinity in IL-33 binding. Indeed, in general, the lengths as well as the sequences of the CDR-like loops of the IL-33 antibody mimics may be randomized during in vitro or in vivo affinity maturation.

For human ¹⁰Fn3 sequences, analyses indicate that, at a minimum, amino acids 1-9, 44-50, 61-54, 82-94 (edges of beta sheets); 19, 21, 30-46 (even), 79-65 (odd) (solvent-accessible faces of both beta sheets); 21-31, 51-56, 76-88 (CDR-like solvent-accessible loops); and 14-16 and 36-45 (other solvent-accessible loops and beta turns) may be randomized to evolve new or improved IL-33-binding proteins. In addition, alterations in the lengths of one or more solvent exposed loops may also be included in such directed evolution methods. Alternatively, changes in the beta-sheet sequences may also be used to evolve new proteins. These mutations change the scaffold and thereby indirectly alter loop structure(s). If this approach is taken, mutations should not saturate the sequence, but rather few mutations should be introduced. No more than 10, 9, 8, 7, 6, 5, 4 3, 2, or 1 amino acid changes may be introduced to the beta-sheet sequences by this approach. The fibronectin type III domain-containing proteins of the invention may lack disulfide bonds.

The IL-33 specific monomers comprising a fibronectin domain/scaffold may be fused to other protein domains. For example, the IL-33 specific monomers comprising a fibronectin domain/scaffold may be integrated with the human immune response by fusing the constant region of an IgG (Fc) with a ¹⁰Fn3 module, possibly through the C-terminus of ¹⁰Fn3. The Fc in such a ¹⁰Fn3-Fc fusion molecule may activate the complement component of the immune response and increases the therapeutic value of the antibody mimic. Similarly, a fusion between a ¹⁰Fn3 and a complement protein, such as Clq may be used, and a fusion between ¹⁰Fn3 and a toxin may be useful. In addition, ¹⁰Fn3 in any form may be fused with albumin to increase its half-life in the bloodstream and its tissue penetration. Any of these fusions may be generated by standard techniques, for example, by expression of the fusion protein from a recombinant fusion gene constructed using publicly available gene sequences.

In addition to fibronectin monomers, any of the fibronectin constructs may be generated as dimers or multimers of ¹⁰Fn3-based antibody mimics as a means to increase the valency and thus the avidity of IL-33 binding. Such multimers may be generated through covalent binding between individual ¹⁰Fn3 modules, for example, by imitating the natural ⁸Fn3-⁹Fn3-¹⁰Fn3 C-to-N-terminus binding or by imitating antibody dimers that are held together through their constant regions. A ¹⁰Fn3-Fc construct may be exploited to design dimers of the general scheme of ¹⁰Fn3-Fc::Fc-¹⁰Fn3. The bonds engineered into the Fc::Fc interface may be covalent or non-covalent. In addition, dimerizing or multimerizing partners other than Fc can be used in ¹⁰Fn3 hybrids to create such higher order structures.

In particular examples, covalently bonded fibronectin multimers may be generated by constructing fusion genes that encode the multimer or, alternatively, by engineering codons for cysteine residues into monomer sequences and allowing disulfide bond formation to occur between the expression products. Non-covalently bonded multimers may also be generated by a variety of techniques. These include the introduction, into monomer sequences, of codons corresponding to positively and/or negatively charged residues and allowing interactions between these residues in the expression products (and therefore between the monomers) to occur. This approach may be simplified by taking advantage of charged residues naturally present in a monomer subunit, for example, the negatively charged residues of fibronectin. Another means for generating non-covalently bonded antibody mimics is to introduce, into the monomer gene (for example, at the amino- or carboxy-termini), the coding sequences for proteins or protein domains known to interact. Such proteins or protein domains include coil-coil motifs, leucine zipper motifs, and any of the numerous protein subunits (or fragments thereof) known to direct formation of dimers or higher order multimers.

The IL-33 specific monomers comprising a fibronectin domain/scaffold may be screened for IL-33 specific binding using a biopanning protocol (Smith & Scott, 1993); IL-33 is biotinylated and the strong biotinstreptavidin interaction is used to immobilize IL-33 on a streptavidin-coated dish. Experiments are performed at room temperature (22° C.). For the initial recovery of phages from a library, 10 μg of a biotinylated IL-33 is immobilized on a streptavidin-coated polystyrene dish (35 mm, Falcon 1008) and then a phage solution (containing about 10¹¹ pfu (plaque-forming unit)) is added. After washing the dish with an appropriate buffer (typically TBST, Tris-HCl (50 mM, pH 7.5), NaCl (150 mM) and Tween 20 (0.5%)), bound phages are eluted by one or combinations of the following conditions: low pH, an addition of a free IL-33, urea (up to 6 M) and, cleaving the IL-33-biotin linker by thrombin. Recovered phages are amplified using the standard protocol using K91kan as the host (Sambrook et al., 1989). The selection process is repeated 3-5 times to concentrate positive clones. From the second round on, the amount of the IL-33 is gradually decreased (to about 1 μg) and the biotinylated IL-33 is mixed with a phage solution before transferring a dish. After the final round, 10 20 clones are picked, and their DNA sequence are be determined. The IL-33 affinity of the clones are measured first by the phage-ELISA method.

To suppress potential binding of the Fn3 framework (background binding) to IL-33, wild-type Fn3 may be added as a competitor in the buffers. In addition, unrelated proteins (e.g., bovine serum albumin, cytochrome c and RNase A) may be used as competitors to select highly IL-33 specific Fn antibody mimics.

The binding affinity of IL-33 specific monomers comprising a fibronectin domain/scaffold on phage surface is characterized semiquantitatively using the phage ELISA technique (Li et al., 1995). Wells of microtiter plates (Nunc) are coated with IL-33 (or with streptavidin followed by the binding of biotinylated IL-33) and blocked with the BLOTTO solution (Pierce). Purified phages (about 10¹⁰ pfu) originating from single plaques (M13)/colonies (fUSE5) are added to each well and incubated overnight at 4° C. After washing wells with an appropriate buffer (see above), bound phages are detected by the standard ELISA protocol using anti-M13 Ab (rabbit, Sigma) and anti-rabbit Ig-peroxidase conjugate (Pierce) or using anti-M13 Ab-peroxidase conjugate (Pharmacia). Colormetric assays are performed using TMB (3,3′,5,5′-tetramethylbenzidine, Pierce). IL-33 specific monomers comprising a fibronectin domain/scaffold are detected using an anti-IL-33 Ab.

After preliminary characterization of IL-33 specific monomers comprising a fibronectin domain/scaffold using phage ELISA, genes are subcloned into the expression vector pEW1. Monomers comprising a fibronectin domain/scaffolds are produced as His-tag fusion proteins and purified, and their conformation, stability and IL-33 affinity are characterized.
IL-33 binding characteristics of IL-33—specific antibodies or monomer/multimer domain polypeptides

IL-33—specific antibodies and monomer/multimer domain polypeptides may have a high binding affinity for an IL-33 polypeptide. IL-33—specific antibodies and monomer/multimer domain polypeptides may have an association rate constant or kₒₙ rate (antibody (Ab)+antigen (Ag)(kₒₙ→Ab−Ag) of at least 10⁵ M⁻¹s⁻¹, at least 1.5×10⁵ M⁻¹s⁻¹, at least 2×10⁵ M⁻¹s⁻¹, at least 2.5×10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹, or 10⁵-10⁸ M⁻¹s⁻¹, 1.5×10⁵ M⁻¹s⁻¹-1×10⁷ M⁻¹s⁻¹, 2×10⁵-1×10⁶ M⁻¹s⁻¹, or 4.5×10⁵ to 5×10⁷ M⁻¹s⁻¹. IL-33—specific antibodies and monomer/multimer domain polypeptides may have a kₒₙ of at least 2×10⁵ M⁻¹s⁻¹, at least 2.5×10⁵ M⁻¹s⁻¹, at least 5×10⁵ M⁻¹s⁻¹, at least 10⁶M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹ as determined by a BIAcore assay and the IL-33—specific antibodies and monomer/multimer domain polypeptides may neutralize human IL-33 in the microneutralization assay. IL-33—specific antibodies and monomer/multimer domain polypeptides may have a kₒₙ of at most 10⁸ M⁻¹s⁻¹, at most 10⁹ M⁻¹s⁻¹, at most 10¹⁰ M⁻¹s⁻¹, at most 10¹¹ M⁻¹s⁻¹, or at most 10¹² M⁻¹s⁻¹ as determined by a BIAcore assay and may neutralize human IL-33 in the microneutralization assay.

IL-33—specific antibodies and monomer/multimer domain polypeptides may have a k_{off} rate (antibody (Ab)+antigen (Ag k_{off} [symbol] Ab−Ag) of less than 10⁻³ s⁻¹, less than 5×10⁻³s⁻¹, less than 10⁻⁴ s¹, less than 2×10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹, or 10⁻³-10⁻¹⁰ s⁻¹, 10⁻⁴-10⁻⁸ s⁻¹, or 10⁻⁵-10⁻⁸ s⁻¹, IL-33—specific antibodies and monomer/multimer domain polypeptides may have a k_{off} of 10⁻⁵ s¹, less than 5×10⁻⁵ s⁻¹, less than 10⁻⁶ s⁻¹, less than 5×10⁻⁶ s⁻¹, less than 10⁻⁷ s¹, less than 5×10⁻⁷ s⁻¹, less than 10⁻⁸ s⁻¹, less than 5×10⁻⁸ s¹, less than 10⁻⁹ s¹, less than 5×10⁻⁹ s⁻¹ or less than 10⁻¹⁰ s⁻¹ as determined by a BIAcore assay and the IL-33—specific antibodies and monomer/multimer domain polypeptides may neutralize human IL-33 in a microneutralization assay. IL-33—specific antibodies and monomer/multimer domain polypeptides may have a k_{off} of greater than 10⁻¹³ s⁻¹, greater than 10⁻¹² s⁻¹, greater than 10⁻¹¹s⁻¹, greater than 10⁻¹⁰ s⁻¹, greater than 10⁻⁹ s⁻¹, or greater than 10⁻⁸ s⁻¹.

IL-33—specific antibodies and monomer/multimer domain polypeptides may have an affinity constant or Kₐ. (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹, or 10²-5×10⁵ M⁻¹, 10⁴-1×10¹⁰ M⁻¹, or 10⁵-1×10⁸ M⁻¹. IL-33—specific antibodies and monomer/multimer domain polypeptides may have a Kₐ of at most 10¹¹ M⁻¹, at most 5×10¹¹ M⁻¹, at most 10¹² M⁻¹, at most 5×10¹² M⁻¹, at most 10¹³ M⁻¹, at most 5×10¹³ M⁻¹, at most 10¹⁴ M⁻¹, or at most 5×10¹⁴ M⁻¹. IL-33—specific antibodies and monomer/multimer domain polypeptides may have a dissociation constant or K_{d} (kₒₙ/k_{off}) of less than 10⁻⁵ M, less than 5×10⁻⁵ M, less than 10⁻⁶ M, less than 5×10⁻⁶ M, less than 10⁻⁷ M, less than 5×10⁻⁷, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³ M, less than 5×10⁻¹³ M, less than 10⁻¹⁴ M, less than 5×10⁻¹⁴ M, less than 10⁻¹⁵ M, or less than 5×10⁻¹⁵ M or 10⁻² M-5×10⁻⁵ M, 10⁻⁶-10¹⁵ M, or 10⁻⁸-10⁻¹⁴ M. IL-33—specific antibodies and monomer/multimer domain polypeptides may have a K_{d} of less than 10⁻⁹ M, less than 5×10⁻⁹M, less than 10⁻¹⁰ M less than 5×10⁻¹⁰ M, less than 1×10⁻¹¹ M, less than 5×10⁻¹¹ M, less than 1×10⁻¹² M, less than 5×10⁻¹² M, less than 10⁻¹³ M, less than 5×10⁻¹³ M or less than 1×10⁻¹⁴ M, or 10⁻⁹ M-10⁻¹⁴ M as determined by a BIAcore assay and the IL-33—specific antibodies and monomer/multimer domain polypeptides may neutralize human IL-33 in the microneutralization assay. IL-33—specific antibodies and monomer/multimer domain polypeptides may have a K_{d} of greater than 10⁻⁹ M, greater than 5×10⁻⁹ M, greater than 10⁻¹⁰ M, greater than 5×10⁻¹⁰ M, greater than 10⁻¹¹ M, greater than 5×10⁻¹¹ M, greater than 10⁻¹² M, greater than 5×10⁻¹² M, greater than 6×10⁻¹² M, greater than 10⁻¹³ M, greater than 5×10⁻¹³ M, greater than 10⁻¹⁴ M, greater than 5×10⁻¹⁴ M or greater than 10⁻⁹ M-10⁻¹⁴ M.

### Therapeutic

The IL-33 binding polypeptides may be used to treat, as a therapeutic, or as treatment for reduction or amelioration of the progression, severity, and/or duration of a disease or disorder (e.g., a disease or disorder characterized by aberrant expression and/or activity of an IL-33 polypeptide, a disease or disorder characterized by aberrant expression and/or activity of an IL-33 receptor or one or more subunits thereof, an autoimmune disease (e.g., lupus, rheumatoid arthritis, and multiple sclerosis), an inflammatory disease (e.g., asthma, allergic disorders, and arthritis), or an infection, or the amelioration of one or more symptoms thereof. In certain embodiments, such terms refer to a reduction in the swelling of organs or tissues, or a reduction in the pain associated with a respiratory condition. In other embodiments, such terms refer to a reduction in the inflammation or constriction of an airway(s) associated with asthma. In other embodiments, such terms refer to a reduction in the replication of an infectious agent, or a reduction in the spread of an infectious agent to other organs or tissues in a subject or to other subjects. In other embodiments, such terms refer to the reduction of the release of inflammatory agents by mast cells, or the reduction of the biological effect of such inflammatory agents.

Therapeutic IL-33 antibodies or monomer/multimer domain polypeptides may inhibit and/or reduce the interaction between the IL-33 polypeptide and the IL-33 receptor (“IL-33R”) or a subunit thereof by approximately 25%, preferably approximately 30%, approximately 35%, approximately 45%, approximately 50%, approximately 55%, approximately 60%, approximately 65%, approximately 70%, approximately 75%, approximately 80%, approximately 85%, approximately 90%, approximately 95%, or approximately 98% relative to a control such as PBS in an in vivo and/or in vitro assay described herein or well-known to one of skill in the art (e.g., an immunoassay such as an ELISA).

Therapeutic IL-33 antibodies or monomer/multimer domain polypeptides may inhibit or reduce the interaction between the IL-33 polypeptide and the IL-33 receptor (“IL-33R”) or one or more subunits thereof by at least 25%, preferably, at least 30%, at least 35%, at least 45%, at least 50%, at least 55%, at least 60%, at least 65%, at least 70%, at least 75%, at least 80%, at least 85%, at least 90%, at least 95%, or at least 98% relative to a control such as phosphate buffered saline (“PBS”) in an in vivo and/or in vitro assay well-known to one of skill in the art. In an alternative embodiment, antibodies or monomer/multimer domain polypeptides that specifically bind to an IL-33 polypeptide do not inhibit the interaction between an IL-33 polypeptide and the IL-33R or one or more subunits thereof relative to a control such as PBS. In another embodiment, antibodies that immunospecifically bind to an IL-33 polypeptide or IL-32 specific monomer/multimer domain polypeptides inhibit the interaction between the IL-33 polypeptide and the IL-33R or one or more subunits thereof by less than 20%, less than 15%, less than 10%, or less than 5% relative to a control such as PBS in vivo and/or in vitro assay well-known to one of skill in the art.

Therapeutic IL-33 antibodies or monomer/multimer domain polypeptides may reduce and/or inhibit proliferation of inflammatory cells (e.g., mast cells, T cells, B cells, macrophages, neutrophils, basophils, and/or eosinophils) by at least 25%, preferably at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS in an in vivo and/or in vitro assay described herein or well-known to one of skill in the art (e.g., a trypan blue assay or ³H-thymidine assay).

In another embodiment, antibodies or monomer/multimer domain polypeptides that immunospecifically bind to an IL-33 polypeptide reduce and/or inhibit infiltration of inflammatory cells into the upper and/or lower respiratory tracts by at least at least 25%, preferably at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS in an in vivo and/or in vitro assay described herein or well-known to one of skill in the art.

Therapeutic IL-33 antibodies or monomer/multimer domain polypeptides may reduce and/or inhibit proliferation of inflammatory cells into the upper and/or respiratory tracts by at least 25%, preferably at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS in an in vivo and/or in vitro assay well known in the art.

Therapeutic IL-33 antibodies or monomer/multimer domain polypeptides may inhibit and/or reduce the expression, activity, serum concentration, and/or release of mast cell proteases, such as chymase and tryptase, by at least 25%, preferably at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS in an in vivo and/or in vitro assay well known to one of skill in the art. Mast cell activity may be measured by culturing primary mast cells or a mast cell line in vitro in the presence of 10 ng/ml of IL-33. Baseline levels of protease (e.g., chymase and tryptase) and leukotriene are determined in the supernatant by commercially available ELISA kits. The ability of antibodies or monomer/multimer domain polypeptides to modulate protease or leukotriene levels is assessed by adding an IL-33-reactive antibody or monomer/multimer domain polypeptide directly to cell cultures at a concentration of 1 μg/ml. Protease and leukotriene levels are assessed at 24 and 36 hour timepoints.

In another embodiment, antibodies or monomer/multimer domain polypeptides that specifically bind to an IL-33 polypeptide inhibit and/or reduce the expression, activity, serum concentration, and/or release of mast cell leukotrienes, such as C4, D4, and E4 by at least 25%, preferably at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay well-known to one of skill in the art.

Furthermore, antibodies or monomer/multimer domain polypeptides that specifically bind to an IL-33 polypeptide inhibit and/or reduce the expression, activity, serum concentration, and/or release of mast cell cytokines, such as TNF-α, IL-4, IL-5, IL-6 or IL-13 by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS in an in vivo and/or in vitro assay well-known to one of skill in the art (e.g., an ELISA or Western blot assay).

Therapeutic IL-33 antibodies or monomer/multimer domain polypeptides may inhibit and/or reduce mast cell infiltration by at least 25%, preferably at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS in an in vivo and/or in vitro assay well-known in the art.

Therapeutic IL-33 antibodies or monomer/multimer domain polypeptides may inhibit and/or reduce infiltration of mast cell precursors in the upper and/or lower respiratory tracts by at least 25%, preferably at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS in an in vivo and/or in vitro assay well-known in the art. In other embodiments, antibodies or monomer/multimer domain polypeptides that specifically bind to an IL-33 polypeptide inhibit and/or reduce proliferation of mast cell precursors by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS in an in vivo and/or in vitro assay well-known to one of skill in the art (e.g., a trypan blue assay, FACS or ³H thymidine assay).

In yet other embodiments, antibodies or monomer/multimer domain polypeptides that specifically bind to an IL-33 polypeptide inhibit and/or reduce airway hyperresponsiveness by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS in an assay well known in the art

Therapeutic IL-33 antibodies or monomer/multimer domain polypeptides may may decrease level of chemokines. Chemokines can include, but are not limited to, XCL1, XCL2, CCL1, CCL2, CCL3, CCL3L1, SCYA3L2, CCL4, CCL4L, CCL5, CCL6, CCL7, CCL8, SCYA9, SCYA10, CCL11, SCYA12, CCL13, CCL14, CCL15, CCL16, CCL17, CCL18, CCL19, CCL20, CCL21, CCL22, CCL23, CCL24, CCL25, CCL26, CCL27, CCL28, clone 391, CARP CC-1, CCL1, CK-1, regakine-1, K203, CXCL1, CXCL1P, CXCL2, CXCL3, PF4, PF4V1, CXCL5, CXCL6, PPBP, SPBPBP, IL8, CXCL9, CXCL10, CXCL11, CXCL12, CXCL14, CXCL15, CXCL16, NAP-4, LFCA-1, Scyba, JSC, VHSV-induced protein, CX3CL1 and fCL1. In some embodiments, modulating the level or activity of the IL-33 polypeptide or a biologically active fragment thereof modulates the physiological effect of a chemokine. Such physiological effect can result from, for example, modulating of the transcription of a nucleic acid encoding a chemokine or from modulating the interaction of the chemokine with its receptor or with another molecule such as a transcription factor. Chemokine receptors can include, but are not limited to, CCR1, CCR2, CCR3, CCR4, CCR5, CCR6, CCR7, CCR8, CCR9, CCR10, CCR11, CXCR1, CXCR2, CXCR3, CXCR4 and CXCR5.

Therapeutic Combinations

The IL-33 specific binding compositions may be used for preventing, managing, treating, and/or ameliorating diseases and disorders including, but not limited to, disorders characterized by aberrant expression and/or activity IL-33, disorders characterized by aberrant expression and/or activity of an IL-33R or one or more subunits thereof, inflammatory disorders, autoimmune disorders, proliferative disorders, or infections, comprising administering to a subject in need thereof an effective amount of one or more antibodies or monomer/multimer domain polypeptides that immunospecifically bind to an IL-33 polypeptide and one or more additional therapies (e.g., prophylactic or therapeutic agents). Additional therapeutic or prophylactic agents include, but are not limited to, small molecules, synthetic drugs, peptides, polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides) antibodies, synthetic or natural inorganic molecules, mimetic agents, and synthetic or natural organic molecules. Additional therapies and therapeutic agents can be found in, e.g., Gilman et al., Goodman and Gilman's: The Pharmacological Basis of Therapeutics, Tenth Ed., McGraw-Hill, New York, 2001; The Merck Manual of Diagnosis and Therapy, Berkow, M. D. et al. (eds.), 17th Ed., Merck Sharp & Dohme Research Laboratories, Rahway, N.J., 1999; and Cecil Textbook of Medicine, 20th Ed., Bennett and Plum (eds.), W.B. Saunders, Philadelphia, 1996. Examples of prophylactic and therapeutic agents include, but are not limited to, immunomodulatory agents, anti-inflammatory agents (e.g., adrenocorticoids, corticosteroids (e.g., beclomethasone, budesonide, flunisolide, fluticasone, triamcinolone, methlyprednisolone, prednisolone, prednisone, hydrocortisone), glucocorticoids, steroids, non-steriodal anti-inflammatory drugs (e.g., aspirin, ibuprofen, diclofenac, and COX-2 inhibitors), and leukotreine antagonists (e.g., montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), anti-viral agents, and antibiotics (e.g., dactinomycin (formerly actinomycin), bleomycin, erythomycin, penicillin, mithramycin, and anthramycin (AMC)).

Any immunomodulatory agent well-known to one of skill in the art may be used. as an additional therapeutic agent Immunomodulatory agents can affect one or more or all aspects of the immune response in a subject. Aspects of the immune response include, but are not limited to, the inflammatory response, the complement cascade, leukocyte and lymphocyte differentiation, proliferation, and/or effector function, monocyte and/or basophil counts, and the cellular communication among cells of the immune system. In some embodiments of the invention, an immunomodulatory agent modulates one aspect of the immune response. In other embodiments, an immunomodulatory agent modulates more than one aspect of the immune response. The administration of an immunomodulatory agent to a subject may inhibit or reduce one or more aspects of the subject's immune response capabilities. In some embodiments of the invention, the immunomodulatory agent may inhibit or suppress the immune response in a subject.

Examples of immunomodulatory agents include, but are not limited to, proteinaceous agents such as cytokines, peptide mimetics (e.g., monomer/multimer domain polypeptides), and antibodies (e.g., human, humanized, chimeric, monoclonal, polyclonal, Fvs, ScFvs, Fab or F(ab)₂ fragments or epitope binding fragments), nucleic acid molecules (e.g., antisense nucleic acid molecules and triple helices), small molecules, organic compounds, and inorganic compounds. In particular, immunomodulatory agents include, but are not limited to, methotrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide), T cell receptor modulators, cytokine receptor modulators, and modulators mast cell modulators.

Examples of T cell receptor modulators include, but are not limited to, anti-T cell receptor antibodies (e.g., anti-CD4 antibodies (e.g., cM-T412 (Boeringer), IDEC-CE9.1.RTM. (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies (e.g., siplizumab (MedImmune, Inc., International Publication Nos. WO 02/098370 and WO 02/069904)), anti-CD11a antibodies (e.g., Xanelim (Genentech)), and anti-B7 antibodies (e.g., IDEC-114) (IDEC))), CTLA4-immunoglobulin, and LFA-3TIP (Biogen, International Publication No. WO 93/08656 and U.S. Pat. No. 6,162,432).

Examples of cytokine receptor modulators include, but are not limited to, soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof), cytokines or fragments thereof (e.g., interleukin IL-2, IL-3, IL-4, IL-5, IL-6, IL-7, IL-8, IL-9, IL-10, IL-11, IL-12, IL-13, IL-15, IL-23, TNF-α, TNF-β, interferon (IFN)-α, IFN-β, IFN-γ, and GM-CSF), anti-cytokine receptor antibodies (e.g., anti-IFN receptor antibodies, anti-IL-2 receptor antibodies (e.g., Zenapax (Protein Design Labs)), anti-IL-3 receptor antibodies, anti-IL-4 receptor antibodies, anti-IL-6 receptor antibodies, anti-IL-10 receptor antibodies, anti-IL-12 receptor antibodies, anti-IL-13 receptor antibodies, anti-IL-15 receptor antibodies, and anti-IL-23 receptor antibodies), anti-cytokine antibodies (e.g., anti-IFN antibodies, anti-TNF-a antibodies, anti-IL-10 antibodies, anti-IL-3 antibodies, anti-IL-6 antibodies, anti-IL-8 antibodies (e.g., ABX-IL-8 (Abgenix)), anti-IL-12 antibodies, anti-IL-13 antibodies, anti-IL-15 antibodies, and anti-IL-23 antibodies).

A cytokine receptor modulator may be IL-3, IL-4, IL-10, or a fragment thereof. A cytokine receptor modulator may be an anti-IL-1β antibody, anti-IL-6 antibody, anti-IL-12 receptor antibody, or anti-TNF-α antibody. A cytokine receptor modulator may be the extracellular domain of a TNF-α receptor or a fragment thereof.

Any anti-inflammatory agent, including agents useful in therapies for inflammatory disorders, well-known to one of skill in the art can be used in the compositions and methods of the invention. Non-limiting examples of anti-inflammatory agents include non-steroidal anti-inflammatory drugs (NSAIDs), steroidal anti-inflammatory drugs, anticholinergics (e.g., atropine sulfate, atropine methylnitrate, and ipratropium bromide (ATROVENT™)), beta2-agonists (e.g., abuterol (VENTOLINM and PROVENTILT™), bitolterol (TORNALATE™), levalbuterol (XOPONEX™), metaproterenol (ALUPENT™), pirbuterol (MAXAIR™), terbutlaine (BRETHAIRE™ and BRETHINE™), albuterol (PROVENTIL™, REPETABS™, and VOLMAX™), formoterol (FORADIL AEROLIZER™), and salmeterol (SEREVENTT™ and SEREVENT DISKUS™)), and methylxanthines (e.g., theophylline (UNIPHYL™, THEO-DUR™, SLO-BID™, AND TEHO-42™)). Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALLEVE™, NAPROSYN™), ketoprofen (ACTRON™) and nabumetone (RELAFEN™). Such NSAIDs function by inhibiting a cyclooxygenase enzyme (e.g., COX-1 and/or COX-2). Examples of steroidal anti-inflammatory drugs include, but are not limited to, glucocorticoids, dexamethasone (DECADRON™), corticosteroids (e.g., methylprednisolone (MEDROL™)), cortisone, hydrocortisone, prednisone (PREDNISONE™ and DELTASONE™), prednisolone (PRELONE™ and PEDIAPRED™), triamcinolone, azulfidine, and inhibitors of eicosanoids (e.g., prostaglandins, thromboxanes, and leukotrienes.

In certain embodiments, an anti-inflammatory agent may be an agent useful in the prevention, management, treatment, and/or amelioration of asthma or one or more symptoms thereof. Non-limiting examples of such agents include adrenergic stimulants (e.g., catecholamines (e.g., epinephrine, isoproterenol, and isoetharine), resorcinols (e.g., metaproterenol, terbutaline, and fenoterol), and saligenins (e.g., salbutamol)), adrenocorticoids, blucocorticoids, corticosteroids (e.g., beclomethadonse, budesonide, flunisolide, fluticasone, triamcinolone, methylprednisolone, prednisolone, and prednisone), other steroids, beta2-agonists (e.g., albtuerol, bitolterol, fenoterol, isoetharine, metaproterenol, pirbuterol, salbutamol, terbutaline, formoterol, salmeterol, and albutamol terbutaline), anti-cholinergics (e.g., ipratropium bromide and oxitropium bromide), IL-4 antagonists (including antibodies), IL-5 antagonists (including antibodies), IL-13 antagonists (including antibodies), PDE4-inhibitor, NF-κB inhibitor, VLA-4 inhibitor, CpG, anti-CD23, selectin antagonists (TBC 1269), mast cell protease inhibitors (e.g., tryptase kinase inhibitors (e.g., GW-45, GW-58, and genisteine), phosphatidylinositide-3′ (PI3)-kinase inhibitors (e.g., calphostin C), and other kinase inhibitors (e.g., staurosporine) (see Temkin et al., 2002 J Immunol 169(5):2662-2669; Vosseller et al., 1997 Mol. Biol. Cell 8(5):909-922; and Nagai et al., 1995 Biochem Biophys Res Commun 208(2):576-581)), a C3 receptor antagonists (including antibodies), immunosuppressant agents (e.g., methotrexate and gold salts), mast cell modulators (e.g., cromolyn sodium (INTAL™) and nedocromil sodium (TILADE™)), and mucolytic agents (e.g., acetylcysteine)). The anti-inflammatory agent may be a leukotriene inhibitor (e.g., montelukast (SINGULAIR™), zafirlukast (ACCOLATE™), pranlukast (ONON™), or zileuton (ZYFLO™).

In certain embodiments, the anti-inflammatory agent may be an agent useful in preventing, treating, managing, and/or ameliorating allergies or one or more symptoms thereof. Non-limiting examples of such agents include antimmediator drugs (e.g., antihistamine, corticosteroids, decongestants, sympathomimetic drugs (e.g., α-adrenergic and β-adrenergic drugs), TNX901 (Leung et al., 2003, N Engl J Med 348(11):986-993), IgE antagonists (e.g., antibodies rhuMAb-E25 omalizumab (see Finn et al., 2003 J Allergy Clin Immuno 111(2):278-284; Corren et al., 2003 J Allergy Clin Immuno 111(1):87-90; Busse and Neaville, 2001 Curr Opin Allergy Clin Immuno 1(1):105-108; and Tang and Powell, 2001, Eur J Pediatr 160(12): 696-704), K-12 and 6HD5 (see Miyajima et al., 2202 Int Arch Allergy Immuno 128(1):24-32), and mAB Hu-901 (see van Neerven et al., 2001 Int Arch Allergy Immuno 124(1-3):400), theophylline and its derivatives, glucocorticoids, and immunotherapies (e.g., repeated long-term injection of allergen, short course desensitization, and venom immunotherapy).

Anti-inflammatory therapies and their dosages, routes of administration, and recommended usage are known in the art and have been described in such literature as the Physician's Desk Reference (57th ed., 2003).

The IL-33—specific antibodies and/or monomer/multimer domain polypeptides may be used to prevent development of asthma in a patent expected to suffer from, or at risk of developing asthma, e.g., patients with genetic disposition for asthma, patients who have or have had one or more respiratory infections, infants, infants born prematurely, children, the elderly, or patients who work with toxic chemicals (i.e., at risk of developing occupational asthma). In some embodiments, the subjects may be children who are at risk of developing asthma, e.g., children who have or have had a respiratory infection, particularly, PIV, RSV, and hMPV, have elevated IgE levels, a family history of asthma, have been exposed to asthma triggers and/or allergens (e.g., animals, cockroach allergens, and tobacco smoke), or have experienced wheezing or bronchial hyperresponsiveness. For a discussion of risk factors for asthma, see, e.g., Klinnert et al., 2001, Pediatrics 108(4):E69; London et al., 2001, Epidemiology, 12(5):577-83; Melen et al., 2001, Allergy, 56(7): 464-52; Mochizuki et al., 2001, J Asthma 38(1):1-21; Arruda et al., 2001, Curr Opin Pulm Med, 7(1):14-19; Castro-Rodriguez et al., 2000, Am J Respir Crit. Care Med 162: 1403-6; Gold, 2000, Environ Health Perspect 108: 643-51; and Csonka et al., 2000, Pediatr Allergy Immuno, 11(4): 225-9.

The IL-33—specific antibodies and/or monomer/multimer domain polypeptides may be used in combination with an effect amount of one or more other therapies to prevent, treat, manage, and/or ameliorate COPD or one or more symptoms thereof. Non-limiting examples of such other therapies include agents such as bronchodilators (e.g. short-acting β2-adrenergic agonist (e.g., albuterol, pirbuterol, terbutaline, and metaproterenol), long-acting β₂-adrenergic agonists (e.g., oral sustained-release albuterol and inhaled salmeterol), anticholinergics (e.g., ipratropium bromide), and theophylline and its derivatives (therapeutic range for theophylline is preferably 10-201 g/mL)), glucocorticoids, exogenous α₁AT (e.g., α₁AT derived from pooled human plasma administered intravenously in a weekly dose of 60 mg/kg), oxygen, lung transplantation, lung volume reduction surgery, endotracheal intubation, ventilation support, yearly influenza vaccine and pneumococcal vaccination with 23-valent polysaccharide, exercise, and smoking cessation.

The IL-33—specific antibodies and/or monomer/multimer domain polypeptides may be used in combination with an effective amount of one or more other therapies to prevent, treat, manage, and/or ameliorate pulmonary fibrosis or one or more symptoms thereof. Non-limiting examples of such therapies include, oxygen, corticosteroids (e.g., daily administration of prednisone beginning at 1-1.5 mg/kg/d (up to 100 mg/d) for six weeks and tapering slowly over 3-6 months to a minimum maintenance dose of 0.25 mg/kg/d), cytotoxic drugs (e.g., cyclophosphamide at 100-120 mg orally once daily and azathioprine at 3 mg/kg up to 200 mg orally once daily), bronchodilators (e.g., short- and long-acting β₂-adrenergic agonists, anticholinergics, and theophylline and its derivatives), and antihistamines (e.g., diphenhydramine and doxylamine).

The IL-33—specific antibodies and/or monomer/multimer domain polypeptides may be used to prevent, treat, manage, and/or ameliorate an autoimmune disorder or one or more symptoms thereof. An effective amount of one or more of the antibodies or monomer/multimer domain polypeptides of the invention may also be administered to a subject to prevent, manage, treat, and/or ameliorate an autoimmune disorder or one or more symptoms thereof in combination with an effective amount another therapy

The autoimmune disorder that may be treated with the IL-33—specific antibody or monomer/multimer domain polypeptide may affect only one organ or tissue type or may affect multiple organs and tissues. Organs and tissues commonly affected by autoimmune disorders include red blood cells, blood vessels, connective tissues, endocrine glands (e.g., the thyroid or pancreas), muscles, joints, and skin. Examples of autoimmune disorders that can be prevented, treated, managed, and/or ameliorated by the methods of the invention include, but are not limited to, adrenergic drug resistance, alopecia greata, ankylosing spondylitis, antiphospholipid syndrome, autoimmune Addison's disease, autoimmune diseases of the adrenal gland, allergic encephalomyelitis, autoimmune hemolytic anemia, autoimmune hepatitis, autoimmune inflammatory eye disease, autoimmune neonatal thrombocytopenia, autoimmune neutropenia, autoimmune oophoritis and orchitis, autoimmune thrombocytopenia, autoimmune thyroiditis, Behcet's disease, bullous pemphigoid, cardiomyopathy, cardiotomy syndrome, celiac sprue-dermatitis, chronic active hepatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, dense deposit disease, discoid lupus, essential mixed cryoglobulinemia, fibromyalgia-fibromyositis, glomerulonephritis (e.g., IgA nephrophathy), gluten-sensitive enteropathy, Goodpasture's syndrome, Graves' disease, Guillain-Barre, hyperthyroidism (i.e., Hashimoto's thyroiditis), idiopathic pulmonary fibrosis, idiopathic Addison's disease, idiopathic thrombocytopenia purpura (ITP), IgA neuropathy, juvenile arthritis, lichen planus, lupus erthematosus, Meniere's disease, mixed connective tissue disease, multiple sclerosis, Myasthenia Gravis, myocarditis, type 1 or immune-mediated diabetes mellitus, myasthenia gravis, myocarditis, neuritis, other endocrine gland failure, pemphigus vulgaris, pernicious anemia, polyarteritis nodosa, polychrondritis, Polyendocrinopathies, polyglandular syndromes, polymyalgia rheumatica, polymyositis and dermatomyositis, post-MI, primary agammaglobulinemia, primary biliary cirrhosis, psoriasis, psoriatic arthritis, Raynauld's phenomenon, relapsing polychondritis, Reiter's syndrome, rheumatic heart disease, rheumatoid arthritis, sarcoidosis, scleroderma, Sjogren's syndrome, stiff-man syndrome, systemic lupus erythematosus, lupus erythematosus, takayasu arteritis, temporal arteristis/giant cell arteritis, ulcerative colitis, urticaria, uveitis, Uveitis Opthalmia, vasculitides such as dermatitis herpetiformis vasculitis, vitiligo, and Wegener's granulomatosis.

The IL-33—specific antibodies and/or monomer/multimer domain polypeptides may be the first, second, third, fourth, or fifth therapy to prevent, manage, treat, and/or ameliorate an autoimmune disorder or one or more symptom thereof. Autoimmune therapies and their dosages, routes of administration and recommended usage are known in the art and have been described in such literature as the Physician's Desk Reference (57th ed., 2003).

### Pharmaceutical Compositions//Administration

To prepare pharmaceutical or sterile compositions including an IL-33 binding agent, the IL-33 binding reagent is mixed with a pharmaceutically acceptable carrier or excipient. Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Selecting an administration regimen for a therapeutic depends on several factors, including the serum or tissue turnover rate of the entity, the level of symptoms, the immunogenicity of the entity, and the accessibility of the target cells in the biological matrix. Preferably, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert, et al. (2003) New Engl. J. Med. 348:601-608; Milgrom, et al. (1999) New Engl. J. Med. 341:1966-1973; Slamon, et al. (2001) New Engl. J. Med. 344:783-792; Beniaminovitz, et al. (2000) New Engl. J. Med. 342:613-619; Ghosh, et al. (2003) New Engl. J. Med. 348:24-32; Lipsky, et al. (2000) New Engl. J. Med. 343:1594-1602).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

IL-33 antibodies, antibody fragments, and monomer/multimer domain polypeptides can be provided by continuous infusion, or by doses at intervals of, e.g., one day, one week, or 1-7 times per week. Doses may be provided intravenously, subcutaneously, topically, orally, nasally, rectally, intramuscular, intracerebrally, or by inhalation. A preferred dose protocol is one involving the maximal dose or dose frequency that avoids significant undesirable side effects. A total weekly dose may be at least 0.05 μg/kg body weight, at least 0.2 μg/kg, at least 0.5 μg/kg, at least 1 μg/kg, at least 10 μg/kg, at least 100 μg/kg, at least 0.2 mg/kg, at least 1.0 mg/kg, at least 2.0 mg/kg, at least 10 mg/kg, at least 25 mg/kg, or at least 50 mg/kg (see, e.g., Yang, et al. (2003) New Engl. J. Med. 349:427-434; Herold, et al. (2002) New Engl. J. Med. 346:1692-1698; Liu, et al. (1999) J. Neurol. Neurosurg. Psych. 67:451-456; Portielji, et al. (20003) Cancer Immunol. Immunother. 52:133-144). The desired dose of a small molecule therapeutic, e.g., a peptide mimetic, natural product, or organic chemical, is about the same as for an antibody or polypeptide, on a moles/kg body weight basis. The desired plasma concentration of a small molecule therapeutic is about the same as for an antibody, on a moles/kg body weight basis. The dose may be at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg. The doses administered to a subject may number at least 1, 2, 3, 4, 5, 6, 7, 8, 9, 10, 11, or 12, or more.

For antibodies, monomer/multimer domain polypeptides, proteins, polypeptides, peptides and fusion proteins specific for IL-33, the dosage administered to a patient may be 0.0001 mg/kg to 100 mg/kg of the patient's body weight. The dosage may be between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight.

The dosage of the antibodies, monomer/multimer domain polypeptides, proteins, polypeptides, peptides and fusion proteins specific for IL-33 may be calculated using the patient's weight in kilograms (kg) multiplied by the dose to be administered in mg/kg. The dosage of the antibodies, monomer/multimer domain polypeptides, proteins, polypeptides, peptides and fusion proteins specific for IL-33 may be 150 μg/kg or less, preferably 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of a patient's body weight.

Unit dose of the antibodies, monomer/multimer domain polypeptides, proteins, polypeptides, peptides and fusion proteins specific for IL-33 may be 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

The dosage of the antibodies, monomer/multimer domain polypeptides, proteins, polypeptides, peptides and fusion proteins specific for IL-33 may achieve a serum titer of at least 0.1 μg/ml, at least 0.5 μg/ml, at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml in a subject. Alternatively, the dosage of the antibodies, monomer/multimer domain polypeptides, proteins, polypeptides, peptides and fusion proteins specific for IL-33 may achieve a serum titer of at least 0.1 μg/ml, at least 0.5 μg/ml, at least 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml in the subject.

Doses of the he antibodies, monomer/multimer domain polypeptides, proteins, polypeptides, peptides and fusion proteins specific for IL-33 may be repeated and the administrations may be separated by at least 1 day, 2 days, 3 days, 5 days, 10 days, 15 days, 30 days, 45 days, 2 months, 75 days, 3 months, or at least 6 months.

An effective amount for a particular patient may vary depending on factors such as the condition being treated, the overall health of the patient, the method route and dose of administration and the severity of side affects (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

The route of administration may be by, e.g., topical or cutaneous application, injection or infusion by intravenous, intraperitoneal, intracerebral, intramuscular, intraocular, intraarterial, intracerebrospinal, intralesional, or by sustained release systems or an implant (see, e.g., Sidman et al. (1983) Biopolymers 22:547-556; Langer, et al. (1981) J. Biomed. Mater. Res. 15:167-277; Langer (1982) Chem. Tech. 12:98-105; Epstein, et al. (1985) Proc. Natl. Acad. Sci. USA 82:3688-3692; Hwang, et al. (1980) Proc. Natl. Acad. Sci. USA 77:4030-4034; U.S. Pat. Nos. 6,350,466 and 6,316,024). Where necessary, the composition may also include a solubilizing agent and a local anesthetic such as lidocaine to ease pain at the site of the injection. In addition, pulmonary administration can also be employed, e.g., by use of an inhaler or nebulizer, and formulation with an aerosolizing agent. See, e.g., U.S. Pat. Nos. 6,019,968, 5,985, 320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety. In one embodiment, an anitbody, combination therapy, or a composition of the invention is administered using Alkermes AIR™ pulmonary drug delivery technology (Alkermes, Inc., Cambridge, Mass.).

If the IL-33—specific antibody or monomer/multimer domain polypeptide is administered in a controlled release or sustained release system, a pump may be used to achieve controlled or sustained release (see Langer, supra; Sefton, 1987, CRC Crit. Ref Biomed. Eng. 14:20; Buchwald et al., 1980, Surgery 88:507; Saudek et al., 1989, N. Engl. J. Med. 321:574). Polymeric materials can be used to achieve controlled or sustained release of the therapies of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. A controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938, PCT publication WO 91/05548, PCT publication WO 96/20698, Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

If the IL-33—specific antibody or monomer/multimer domain polypeptide is administered topically, it can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the IL-33—specific antibody or monomer/multimer domain polypeptide is administered intranasally, it can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

Methods for co-administration or treatment with a second therapeutic agent, e.g., a cytokine, steroid, chemotherapeutic agent, antibiotic, or radiation, are well known in the art (see, e.g., Hardman, et al. (eds.) (2001) Goodman and Gilman's The Pharmacological Basis of Therapeutics, 10.sup.th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila. Pa.). An effective amount of therapeutic may decrease the symptoms by at least 10%; by at least 20%; at least about 30%; at least 40%, or at least 50%.

Therapies (e.g., prophylactic or therapeutic agents), other than IL-33—specific antibodies or monomer/multimer domain polypeptides which can be administered in combination with the IL-33—specific antibodies or monomer/multimer domain polypeptides may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart from the IL-33—specific antibodies or monomer/multimer domain polypeptides. The two or more therapies may be administered within one same patent visit.

The antibodies IL-33—specific antibodies or monomer/multimer domain polypeptides and the other therapies may be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

All publications, patents and patent applications mentioned in this specification are herein incorporated by reference into the specification to the same extent as if each individual publication, patent or patent application was specifically and individually indicated to be incorporated herein by reference.

This application claims priority to and incorporates by reference application Ser. No. 60/924,542 filed May 18, 2007 and 61/064,167 filed Feb. 20, 2008.

The set of examples that follow are provided for the purpose of illustration only and the invention should in no way be construed as being limited to these examples.

### EXAMPLES

### Example 1

### IL-33 Induces Cytokine and Leukotriene Production from Mouse BMMCs

Mouse Mast Cells: Mouse bone marrow derived mast cells, (BMMCs) were differentiated in the presence of SCF and IL-3, as previously described. At 5 weeks, mast cell maturity was determined to be 95% by FACs double staining for IgE receptor and cKit.

Stimulation of Mouse Mast Cells: BMMCs were incubated with IL-33 (1-100 ng/ml) to induce cytokine and leukotriene production. For IgE receptor cross-linking, BMMCs were incubated for 4 hours at 37° C. with anti-DNP IgE (Sigma), then stimulated 24 h with 30 ng/ml DNP-BSA. For TLR4 and TLR2 activation, BMMCs were stimulated with ultra pure E. coli LPS and Pam3CSK4 (Invivogen).

ELISAs: Mouse IL-6, human IL-5, human IL-13, and human TNF alpha concentrations in culture supernatants were measured using R&D ELISA kits. PGD2, PGE2, and Cysteinyl Leukotrienes levels were determined using Cayman Chemical kits. Concentrations of additional cytokines and chemokines were determined by Perbio Searchlight service. All samples were assayed in triplicate.

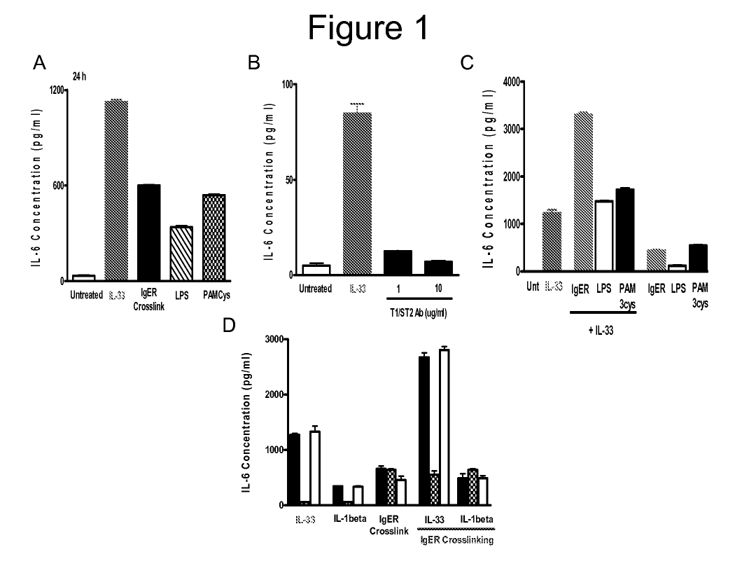

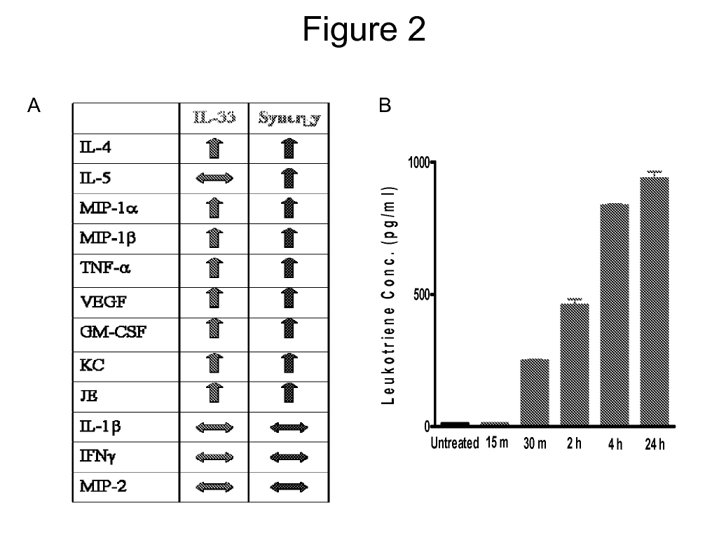

Results: IL-33 induces production of numerous cytokines and of leukotrienes by mouse BMMCs. See FIG. 1A-C which shows the induction of IL-6 production from mouse BMMCs by IL-33. Note that other stimuli, such as IgE receptor cross-linking, and LPS and PAMcys (TLR agonists) failed to induce as great a response in IL-6 expression as IL-33. See also FIG. 2A, which indicates that IL-33 induces, inter alia, IL-4 and GM-CSF production. IL-6 production from mouse BMMCs was specifically due to IL-33; IL-6 production by mouse BMMCs was abrogated by T1/ST2 (an IL-33 receptor) antibody.

IL-33 induced production of various other inflammatory mediators in the mouse BMMCs. See FIG. 2A, which indicates that IL-33 induced production of chemokines, e.g., MIP-1α, MIP-1β, MIP2, by the mouse BMMCs. See FIG. 2B, which charts a time-dependent induction of cysteinyl leukotrienes by IL-33 activation.

Furthermore, IL-33 appeared to act in synergy with IgE receptor cross-linking of mast cells by stimulating mediator release more than three-fold. See FIG. 1C, which quantitatively shows synergistic induction of IL-6 production by mouse BMMCs (compare IL-6 induction by IL-33+ IgE receptor cross-linking to either of IL-33 or IgE receptor cross-linking alone). See also FIG. 2A which shows that IL-33 acts in synergy with IgE receptor in stimulating release of IL-4, IL-5, MIP-1α, MIP-1β, TNF-α, VEGF, GM-CSF, KC, and JE from mouse BMMCs.

Further, IL-33 signaling is MyD88 (MyD88 is a TLR signaling adaptor protein) dependent. See FIG. 1D.

### Example 2

### IL-33 does not Appear to Induce Degranulation in Mouse BMMCs

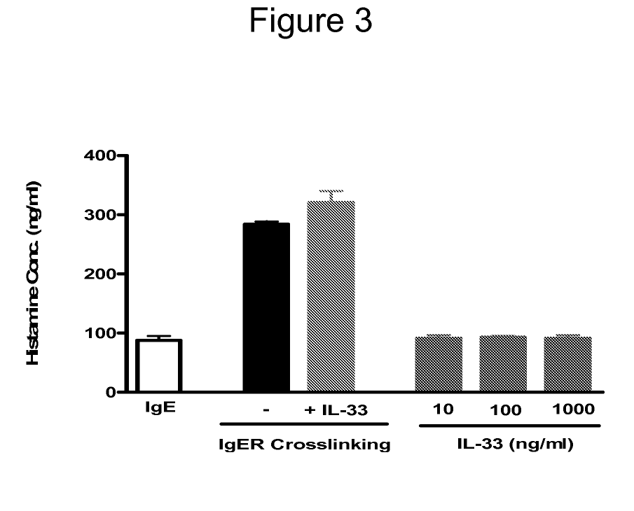

To determine whether IL-33 induced degranulation in mouse BMMCs, mouse BMMCs were stimulated with IL-33 for 30 min and culture supernatants were assayed for histamine release. IL-33 (10-1000 ng/ml) alone did not induce histamine production from BMMCs. See FIG. 3, which shows that 10, 100, and 1000 ng/ml concentrations of IL-33 did not induce histamine release. Furthermore, it appeared that the combination of IL-33 and IgE receptor cross-linking failed to induce histamine production. Again, see FIG. 3, which shows near equivalent histamine release in cells stimulated by IL-33+IgE receptor cross-linking relative to IgE receptor cross-linking alone.

### Example 3

### IL-33 Induces AHR in Naïve Mice

AHR Induction: BALB/c mice were treated intranasally (i.n.) with IL-33, IL-13 or PBS to induce AHR.

Assessment of AHR: AHR was assessed 4 or 24 h following treatment. AHR was determined in spontaneously breathing animals by measuring Penh responses (Buxco Electronics, Conn.) to inhaled methacholine (3-300 mg/ml for 1 min, Penh values were averaged over 5 min recordings/dose). Penh results were confirmed by measuring airways resistance and compliance in anaesthetized and mechanically ventilated mice in response to inhaled methacholine (3-100 mg/ml, results were averaged over 3 min recordings/dose) using a Buxco whole body plethysmography system.

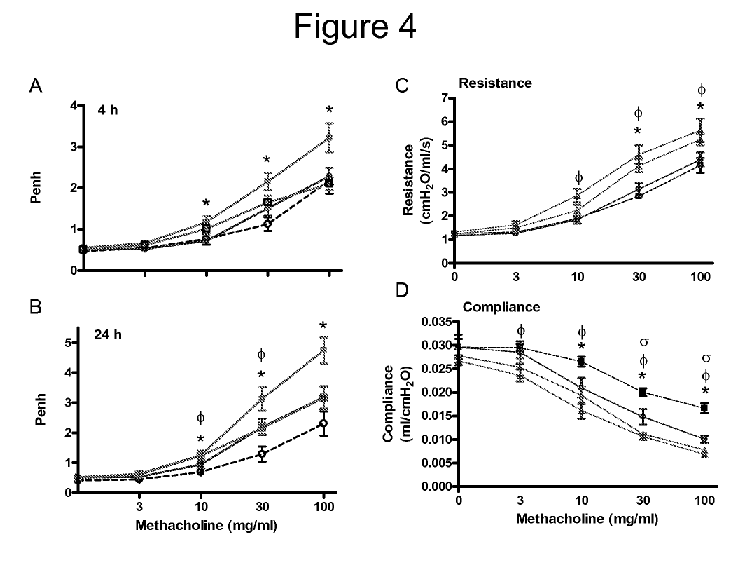

Results: IL-33 induces AHR in naïve mice. As shown in FIG. 4, mice treated with IL-33 have elevated Penh responses 4 hr (FIG. 4A) and 24 hr (FIG. 4B) post administration. The IL-33 treated mice also have increased resistance (FIG. 4C) and decreased compliance (FIG. 4D).

### Example 4

### IL-33 Induces Cytokine and Mucin Expression in Lungs of Naïve Mice

Method: BALB/c mice were treated i.n. with IL-33, IL-13, or PBS and lung tissue taken 24 h later. mRNA levels in lung tissue were determined by quantitative RT-PCR.

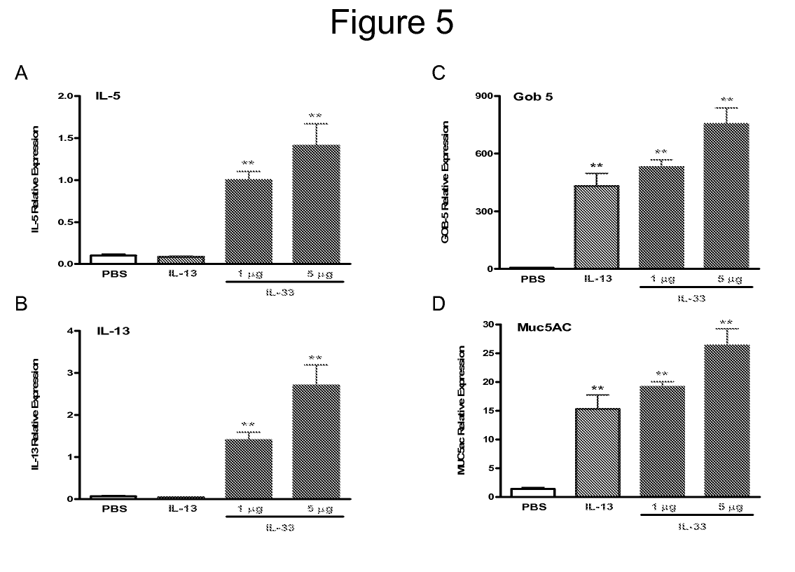

Results: Lungs of mice treated with IL-33 exhibited much higher IL-5 (FIG. 5A) and IL-13 (FIG. 5B) mRNA expression levels than lungs of mice treated with IL-13 or PBS. In fact, the lungs of IL-13 and PBS control treated mice exhibited similar levels of IL-5 (FIG. 5A) and IL-13 (FIG. 5B) induction. Furthermore, mucin gene expression levels were induced to a greater extent in lungs of IL-33 treated mice relative to IL-13 and PBS treated mice. See FIGS. 5C and 5D which show expression levels of Gob 5 and Muc5AC, respectively, in the lungs of the mice.

### Example 5

### IL-33 Directly Activates Macrophages in Mouse Lung Tissue and Serum

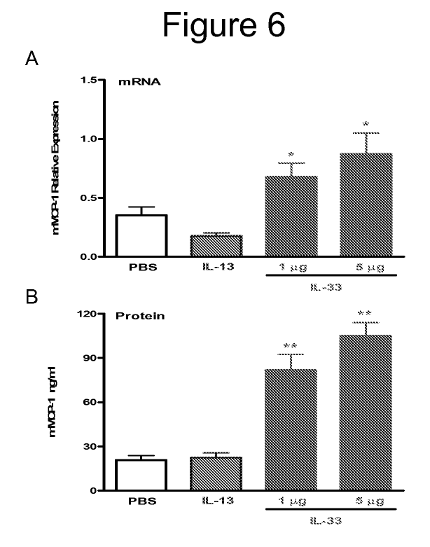

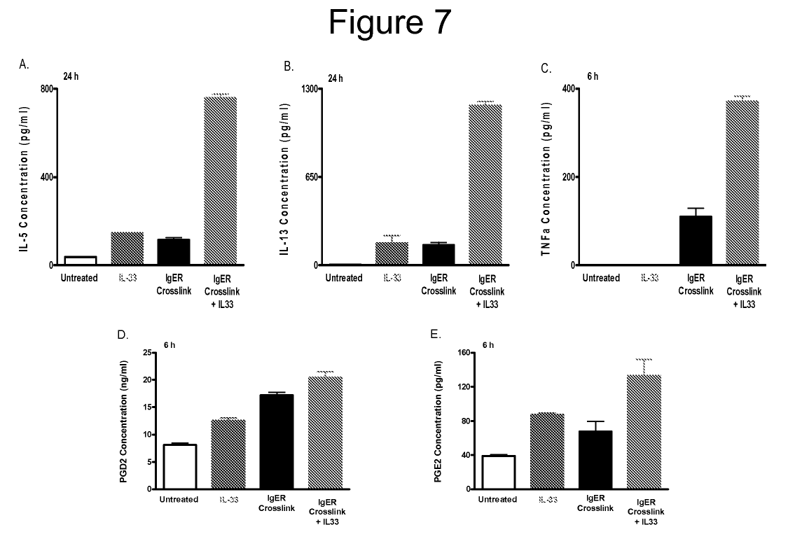

BALB/c mice were treated i.n. with IL-33, IL-13, or PBS. mMCP-1 levels were determined 24 h later via mRNA levels in lung tissue and ELISA in serum. As seen in FIG. 6, mMCP-1 levels were elevated in lung (FIG. 6A) and serum (FIG. 6B) following administration. These levels of elevation were not observed in PBS or IL-13 treated mice. In fact, mice treated with IL-13 exhibited similar or lower levels of mMCP-1 expression in lung (FIG. 6A) and serum (FIG. 6B) compared to PBS treated controls.

### Example 6

### IL-33 Activates Human Mast Cells

Human Mast Cells: Human cord blood derived mast cells were obtained as has been previously described. Cells were harvested when >95% stained positively with toluidine blue, and were further cultured with stem cell factor (SCF, 100 ng/ml, R & D Systems) and IL-4 (10 ng/ml, R & D Systems) for 4 days at 37° C. and 5% CO₂. On day 4, cells were incubated overnight with IgE (10 ug/ml; Chemicon), washed and plated. Cells were then stimulated with rabbit anti-human IgE antibody (1 ug/ml; ICN biomedicals), rhIL-33 (10 ng/ml; Axxora, LLC) or in combination at various times.

IL-33 stimulation of HMCs induced the production of cytokines IL-5 (FIG. 5A) and IL-13 (FIG. 5B) when compared to untreated cells. Interestingly, IL-33+IgE receptor cross-linking significantly enhanced the production of IL-5 (FIG. 5A), IL-13 (FIG. 5B), and TNF-alpha (FIG. 5C) from these cells. IL-33 also induced production of other inflammatory mediators PGD2 (FIG. 5D) and PGE2 (FIG. 5E). Thus, IL-33 activates human cord blood derived mast cells. These findings illustrate IL-33's potential role in activating mast cells involved in allergic disease.

### Example 7

### IL-33 is a Potent Activator of Mouse Bone Marrow Derived Mast Cells

The effect of IL-33 stimulation on mouse bone marrow derived mast cells (BMMCs) was assessed. BMMCs were obtained from Balb/c (Harlan), C57/BL6-wild type and C57/BL6J Kit^{W-sh/W-sh} mice (Grimbaldeston, M. A. et al. Mast Cell-Deficient W-sash c-kit Mutant Kit^{W-sh/W-sh} Mice as a Model for Investigating Mast Cell Biology in Vivo. Am J Pathol 167, 835-848 (2005)) that were housed at the laboratory animal research facility at Medlmmune, Inc and treated according to protocols for animal care established and approved by the IACUC at Medimmune, Inc. MyD88 (Adachi, O. et al. Targeted Disruption of the MyD88 Gene Results in Loss of IL-1- and IL-18-Mediated Function. Immunity 9, 143-150 (1998)) and TRIF (Yamamoto, M. et al. Role of Adaptor TRIF in the MyD88-Independent Toll-Like Receptor Signaling Pathway. Science 301, 640-643 (2003)) knock-out mice were obtained from Dr. S Akira (Osaka University, Osaka, Japan) and backcrossed to C57BL/6 mice for at least six generations. MyD88 knock-out and TRIF knock-out mice were maintained under specific pathogen-free conditions at University of Massachusetts Medical School, Worcester, Mass.

Mouse BMMCs were differentiated as previously described (Mekori, Y. A., Oh, C. K., & Metcalfe, D. D. IL-3-dependent murine mast cells undergo apoptosis on removal of IL-3. Prevention of apoptosis by c-kit ligand. J Immunol 151, 3775-3784 (1993)). In brief, the bone marrow was flushed from the femurs of euthanized Balb/c, C57BL/6 mice (wild-type (WT) control), MyD88 knock-out, or TRIF knock-out mice with RPMI media. The bone marrow cell culture was established at a density of 5×10̂5 cells/ml in RPMI medium supplemented with 10% FBS, 1% Penicillin Streptomycin, 25 ng/ml Stem Cell Factor (SCF)(R&D Systems) and 10 ng/ml IL-3 (R&D). Cells were maintained at 37° C. and nonadherent cells were passaged every 3-4 days to select for developing mast cells. At 5 weeks, the BMMC culture was found to be 95% pure (c-kit⁺FcERI⁺) by flow cytometric analysis. Cells were phenotyped using flow cytometric analysis as previously described (Kearley, J., Barker, J. E., Robinson, D. S., & Lloyd, C. M. Resolution of airway inflammation and hyperreactivity after in vivo transfer of CD4+CD25+regulatory T cells is interleukin 10 dependent. J. Exp. Med. 202, 1539-1547 (2005)). Briefly, cells were stained in cold FACS buffer (PBS containing 1% FCS, 0.1% sodium azide). Non-specific binding was blocked with Fc Block (BD Biosciences) prior to antibody staining Antibodies used were anti-mouse CD4 (BD Biosciences), c-kit (BD Biosciences), FcERI (eBiosciences), and T1/ST2 (MD Biosciences) and their relevant isotype controls. Samples were analyzed using an LSRII flow cytometer and FACS DIVA software (BD Biosciences). Results were further analyzed using FlowJo (TreeStar Corp.)

For IL-33 activation, cells were incubated with the indicated concentrations of recombinant IL-33 (Axxora, LLC) for 24 h. For IgER crosslinking, BMMCs were incubated for 4 hours at 37° C. with anti-DNP IgE (Sigma) then stimulated for 24 h with 30 ng/ml DNP-BSA. For TLR4 and TLR2 activation, BMMCs were stimulated with ultra pure E. coli LPS and Pam3CSK4 respectively (Invitrogen) for 24 h. In certain studies, T1/ST2 antibody (MD Biosciences) was incubated with BMMCs for 30 minutes prior to addition of IL-33.

Mouse IL-6 was measured using an ELISA kit according to the manufacturer's protocol (R&D systems). PGD2, PGE2, and Cysteinyl leukotrienes were determined in BAL supernatant using kits from Cayman Chemical according to the manufacturer's protocol. Mouse mast cell protease-1 concentrations were determined in serum using a Moredun Scientific ELISA kit. Concentrations of additional cytokines and chemokines were determined by Perbio Searchlight service. All samples were assayed in triplicate.

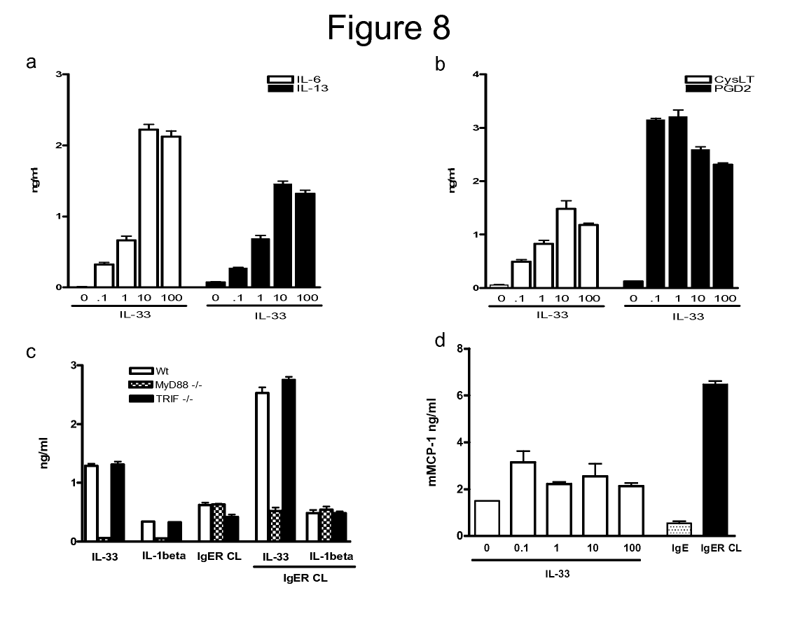

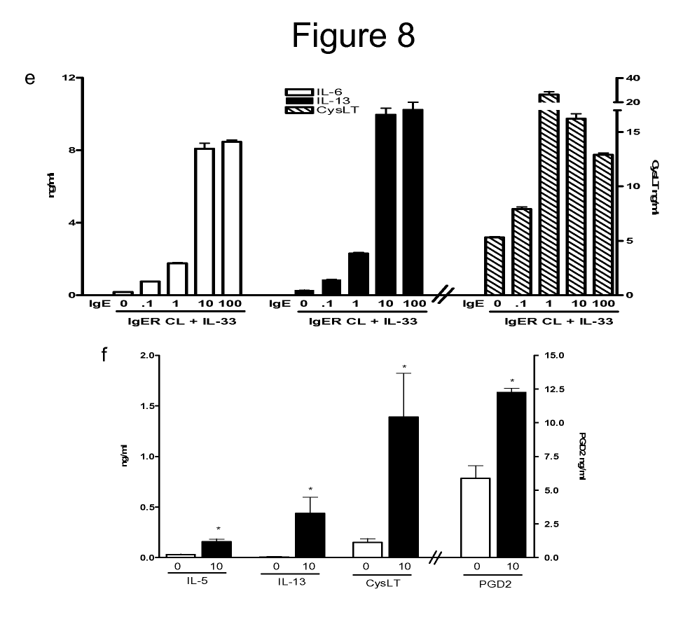

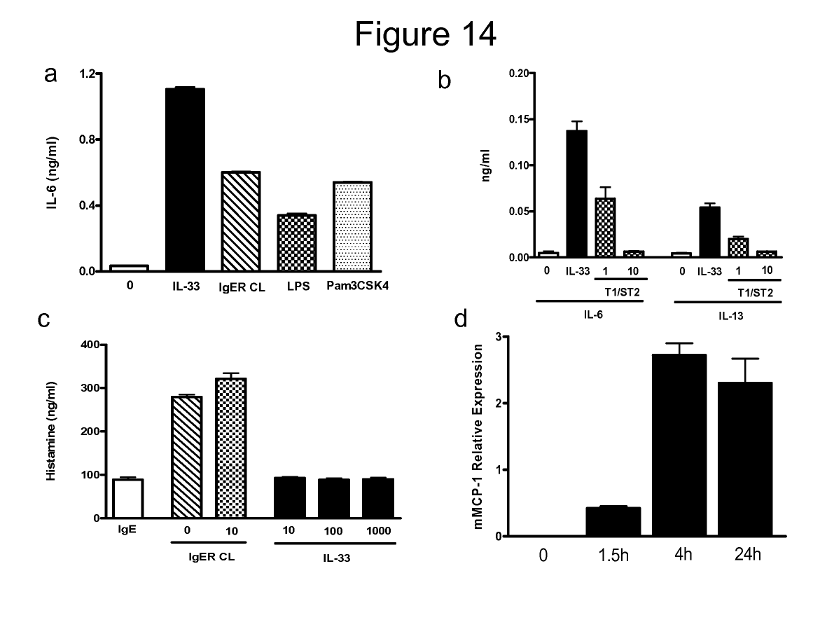

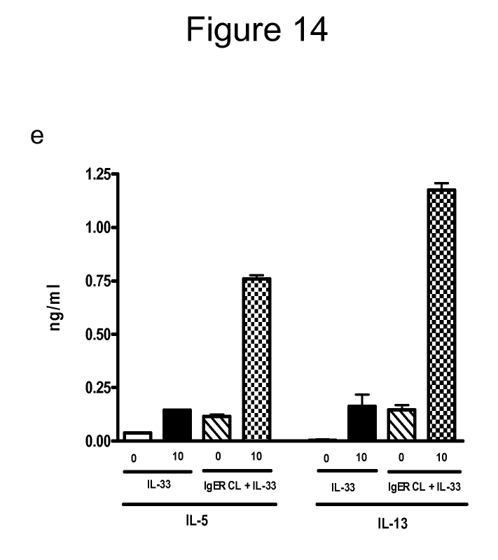

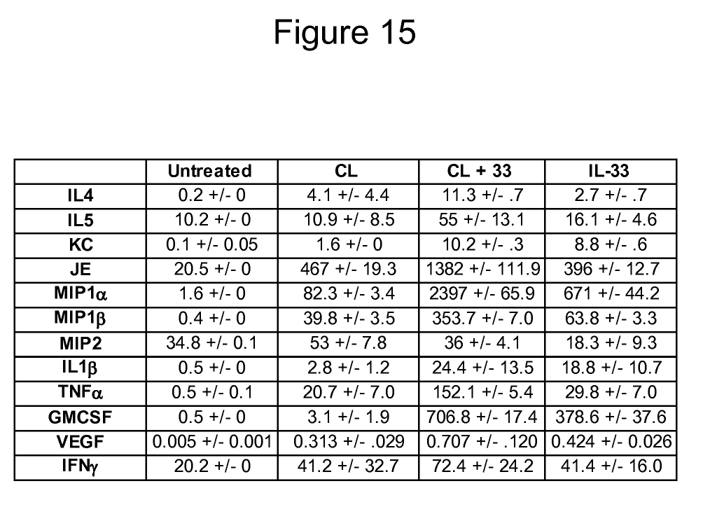

IL-33 was found to be a potent activator of BMMCs inducing an array of cytokines and pro-inflammatory mediators (FIG. 8 and FIG. 15). Among those examined in detail, IL-33 induced a dose related increase in IL-6 (FIG. 8a), the Th2 cytokines IL-4, IL-5 (FIG. 15) and IL-13 (FIG. 8a) and the eicosanoids, PGD₂ and cysteinyl leukotrienes (FIG. 8b). In fact, IL-33 induced greater levels of these cytokines when compared to other stimuli, i.e. high affinity IgE receptor crosslinking, LPS (TLR4 activation) or the synthetic ligand Pam₃CSK4 (TLR2 activation, FIG. 14a). Further, these effects were T1/ST2 dependent as a neutralizing antibody to this receptor blocked cytokine production (FIG. 14b). T 1/ST2 belongs to the TIR family of receptors, members of which signal via the adapter molecules MyD88 and TRIF.

To investigate through which of the adapter molecules IL-33 signals, BMMC from MyD88 deficient, TRIF deficient and C57BL/6 wildtype mice were differentiated and stimulated accordingly. Increased and comparable levels of IL-6 and IL-13 were detected in C57BL/6 and TRIF deficient BMMCs but not in cells from MyD88−/− animals suggesting that IL-33 signaling is MyD88 dependent (FIG. 8c). See also Example 1.

### Example 8

### IL-33 does not Appear to Induce Histamine Release but Acts in Synergy with IgE Receptor Activation to Enhance Cytokine Production

Given that IL-33 stimulated cytokine production, it was determined whether it would induce mast cell degranulation. BMMCs were stimulated for 30 min and the supernatants analyzed for histamine. In these experimental conditions, IL-33 (10-100 ng/ml) failed to induce histamine release when compared to IgE receptor cross linking (FIG. 14c). However, IL-33 did induce mouse mast cell protease 1, (mMCP-1), a mast cell granule associated protease, at both the level of mRNA expression (FIG. 14d) and protein (FIG. 8d).

Previous in vitro studies have shown that IL-1 family members can enhance IgE receptor mediated activation. Here IL-33 was demonstrated to have a synergistic effect significantly enhancing cytokine and eicosanoid production (approximately 4-8 fold when compared to IL-33 or IgE receptor activation alone) from IgE receptor activated BMMCs (FIG. 8e (shown IL-5, IL-13, cysteinyl leukotrienes (Cys LT) and PGD₂) and FIG. 15). See also Example 2.

### Example 9

### IL-33 Induces a Similar Pro-Inflammatory Profile in Human Mast Cells

The effects of IL-33 on human cord blood derived mast cells (HMCs) was also determined. Human cord blood derived mast cells were obtained as previously described (Ochi, H. et al. T Helper Cell Type 2 Cytokine mediated Co-mitogenic Responses and CCR3 Expression During Differentiation of Human Mast Cells In Vitro. J. Exp. Med. 190, 267-280 (1999)). In brief, heparin-treated umbilical cord blood was obtained from placentas after routine caesarean section deliveries. After dextran sedimentation of the blood, mononuclear cells were obtained by centrifugation of the buffy coats through a cushion of Ficoll-Hypaque® (1.77 g/ml; Pharmacia). Residual erythrocytes were removed by hypotonic lysis, and the mononuclear cells were suspended in RPMI 1640 (Invitrogen) supplemented with 10% fetal bovine serum (Sigma), 2 mM L-glutamine, 0.1 mM nonessential amino acids, 0.2 μM 2-ME, 100 U/ml penicillin, 100 μg/ml streptomycin, and 2 μg/ml gentamycin. The cell suspensions were seeded at a density of 10⁶ cells/ml and cultured in the presence of 100 ng/ml SCF, 50 ng/ml IL-6, and 10 ng/ml IL-10. Cells were harvested when >95% stained positively with toluidine blue, and were further cultured with stem cell factor (SCF, 100 ng/ml, R & D Systems) and IL-4 (10 ng/ml, R & D Systems) for 4 days at 37° C. and 5% CO₂. On day 4, cells were incubated overnight with IgE (10 ug/ml; Chemicon), washed and plated. Cells were then stimulated with rabbit anti-human IgE antibody (1 ug/ml; ICN biomedicals), rhIL-33 (10 ng/ml; Axxora, LLC) or in combination at various times. Human IL-5, human IL-13, and human TNFα were measured using ELISA kits according to the manufacturer's protocol (R&D systems).

A cytokine profile almost identical to that of mouse BMMCs was observed; increases in the TH2 cytokines, IL-5 and IL-13, and in particular the eicosanoids, PGD₂ and Cys LT (FIG. 8f) was observed. Further, IL-33-induced cytokine, but not eicosanoid production, was enhanced with IgE receptor activation (FIG. 14e). Similarly, IL-33 did not induce degranulation in HMCs. See also Example 6.

### Example 11

### IL-33 Induces AHR and Inflammation in Naive Mice

Because IL-33 activates mast cells and induces a TH2 cytokine profile in vitro (FIG. 8), the effect of IL-33 in the airways of naive mice was investigated. IL-33 (5 μg per dose) was administered intranasally to BALB/c mice on days 1 through 3 and the airway responses to inhaled methacholine measured 24 h later (on days 2 and 4). Mice were anaethetised using inhaled isofluorane, and 5 ug recombinant IL-33 or the equivalent volume of PBS was administered intranasally. Mice were dosed once, twice or 3 times, with 24 hours interval between each dose. AHR and lung inflammation was assessed 24 hours after the final IL-33 dose. In some studies, mice were treated with 0.5 mg of a depleting antibody against CD4 (GK1.5 (Gavett, S. H., Chen, X., Finkelman, F., & Wills-Karp, M. Depletion of murine CD4+T lymphocytes prevents antigen-induced airway hyperreactivity and pulmonary eosinophilia. Am J Respir Cell Mol Biol 10, 587-593 (1994)) or control Ig 24 hours prior to the first dose of IL-33.

Hyperreactivity was determined by an adapted version of previously described methods (Wagers, S. S. et al. Intrinsic and antigen-induced airway hyperresponsiveness are the result of diverse physiological mechanisms. J Appl Physiol 102, 221-230 (2007)). Briefly, mice were anesthetized with pentobarbital sodium and connected to a small animal ventilator (FlexiVent, Scireq, Montreal, Calif.). Baseline mechanical ventilation was applied at 180 breaths/min with a tidal volume of 0.25 ml against a positive end-expiratory pressure of 3 cmH₂O. At the start of the experiment, a standard lung volume history was established by delivering two deep lung inflations of 1 ml followed by 2 min of regular ventilation. Next baseline recordings of all parameters (Snapshot and Quick-Prime) were obtained. The mice were then challenged with an aerosol of PBS (40 s) achieved by directing the inspiratory flow from the ventilator through an ultrasonic nebulizer. Recordings of all parameters were made every 10 s for 3 mins, alternating between signals (Snapshot and Quick-Prime, the latter measurements being made by interrupting the ventilation for a 1 s passive expiration followed by 2 s broadband (1-19.625 Hz) volume pertubation. The peak to peak excusion of the ventilator piston during delivery of these pertubations was 0.17 ml above the functional residual capacity, resulting in a volume delivered of 0.14 ml after accounting for gas compression in the ventilator cylinder and connecting tubing). Finally 2 more deep lung inflations were given and the above protocol repeated 3 more times with aerosols containing methacholine (Sigma) at sequentially increasing concentrations of 3.125, 12.5 and 50 mg/ml.

Parameters obtained when using the single compartment linear model: Snapshot signals

P(t)=RV(t)+EV(t)+Pₒ

R=Dynamic Resistance (i.e. level of constriction in the lungs)

E=Dynamic Elastance (i.e. elastic rigidity of the lungs)

C=Compliance=1/E (i.e. Ease with which the lungs can be extended)

COD: coefficient of determination (quality control parameter, goodness of model fit)

Parameters derived from the constant phase model: Quick-Prime signals

$$Zin ( f ) = R + i 2 π fl + {G}_{t} - {{iH}_{t} ( 2 π f )}^{α}$$

R=Newtonian Resistance, Rₙ (i.e. represents resistance of central airways)

I=Inertance (inertive properties of gases in the central airways, negligible in mice at >20 Hz

G=Tissue dampening or closely related to tissue resistance

H=Tissue elastance

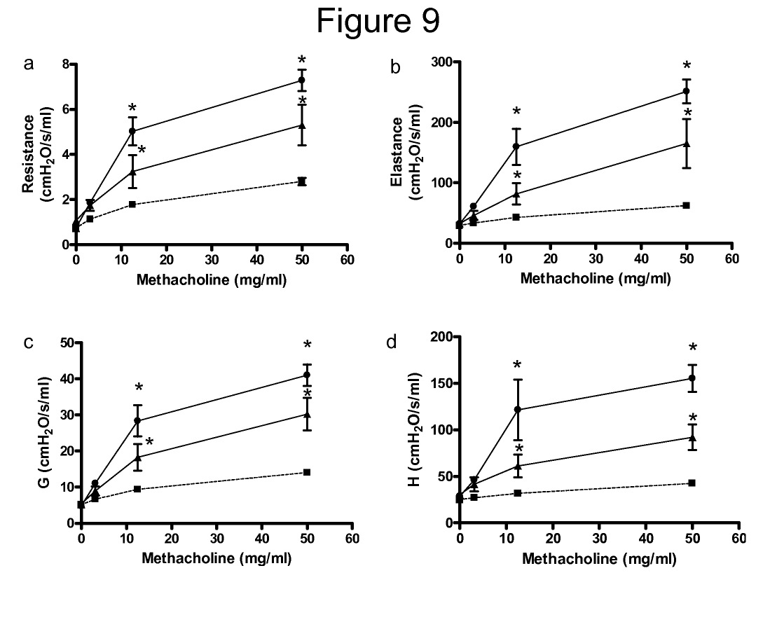

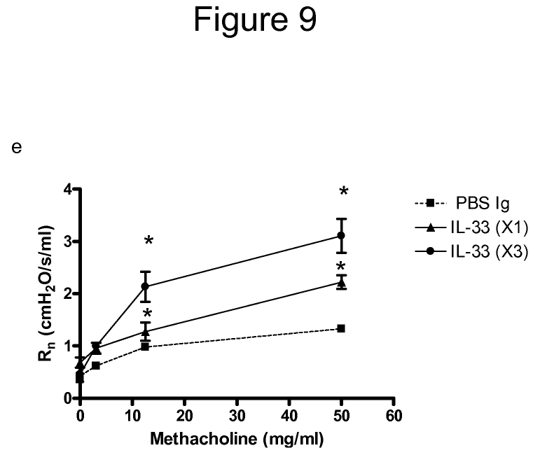

Surprisingly, a single administration of IL-33 induced a significant AHR in naïve mice and this was further enhanced after 3 challenges when compared to PBS alone (FIG. 9). Interestingly, IL-33 induced significant changes in all lung function parameters examined. Using the flexivent lung mechanics system increases in total lung resistance (FIG. 9a) and elastance (FIG. 9b) were observed as well as in the tissue parameters G (tissue resistance, FIG. 9c) and H (tissue elastance, FIG. 9d). Further marked increases in newtonian resistance (Rn, FIG. 9e), a parameter which reflects resistance of the central airways and is related to airway smooth muscle contraction were also observed. Notably, one dose of IL-33 was significantly more effective at inducing AHR than the same amount of the TH2 cytokine IL-13, a well known inducer of AHR in naïve mice (data not shown).

Further examination found that IL-33 induced a dose and time dependent inflammation in the airways of mice. Airway inflammation was examined by assessing airway lumen and lung tissue of the mice. (i) Airway lumen. After lung function measurements had been made, mice were bled by cardiac puncture. BAL was performed with 3×0.6 ml aliquots of HBSS containing 10 mM HEPES and EDTA via a tracheal cannula. BAL fluid was centrifuged (400 g, 4° C.) and the cells removed. Total cell counts were determined using a coulter Z2 particle counter (Beckman Coulter Corporation) and differential cell counts (of at least 500 cells per slide) were performed on cytospin preparations stained with eosin and methylene blue (Diff-Quik, Dade Diagnostics). (ii) Lung tissue. To disaggregate the cells from the lung tissue, one lobe (≈100 mg) of lung was incubated at 37° C. for 1 h in digest reagent (1.8 mg/ml Liberase (Blenzyme 2; Roche), 25 μg/ml DNase (type 1; Roche)) in RPMI/10% FCS. The recovered cells were filtered through a 70-μm nylon sieve (Falcon), washed twice, resuspended in RPMI/10% FCS, and counted using a Coulter Z2 particle counter. Cytocentrifuge preparations were prepared and stained, and differential counts were performed as for BAL.

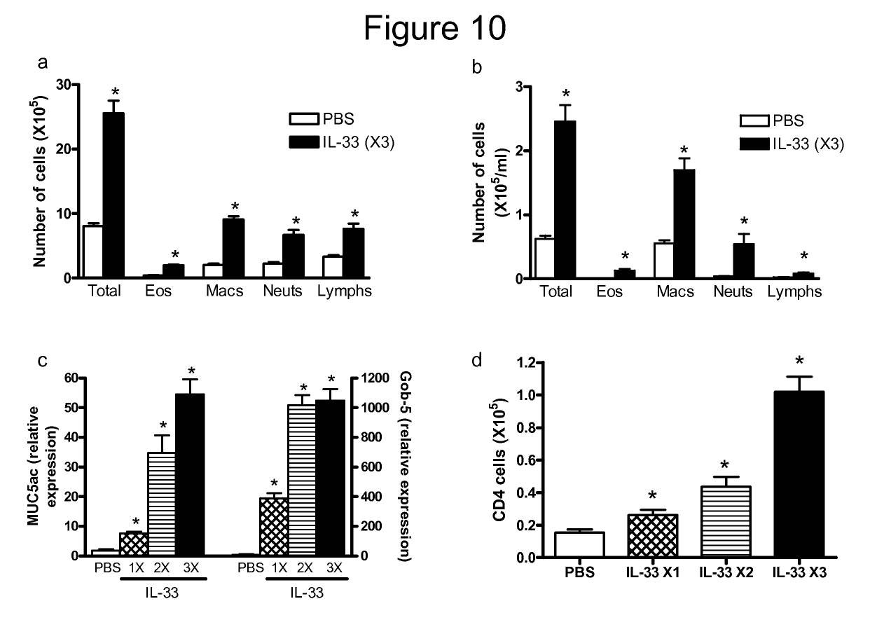

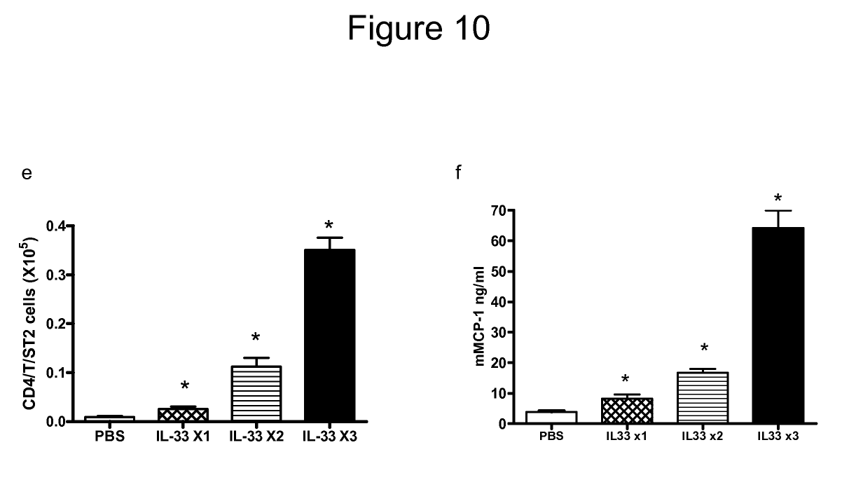

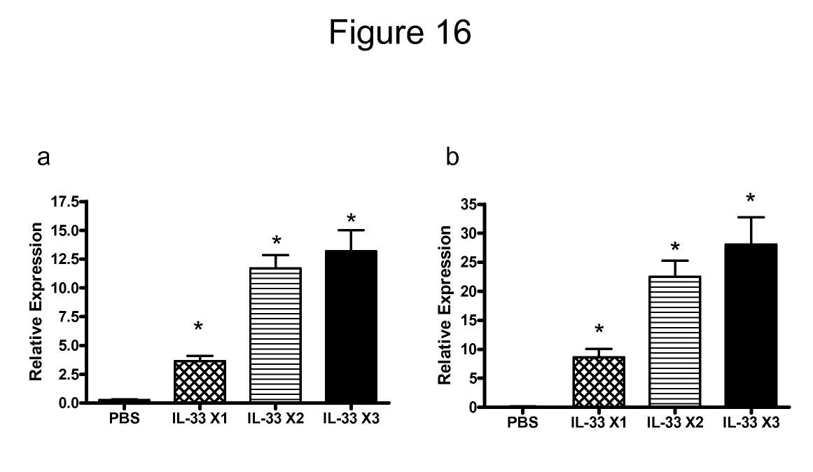

One administration of IL-33 induced significant increases in mRNA expression for the mucin genes Gob-5 and MUC5AC (FIG. 10c) and the Th2 cytokines, IL-5 and IL-13 (FIG. 16 a, b respectively), although there was no inflammation or mucus production visible at this time in lung sections. However, after 3 doses of IL-33, there was marked inflammation at the level of protein and cellular recruitment in BAL; increases in macrophages, lymphocytes, eosinophils and neutrophils were observed (FIG. 10a). In addition, Gob-5 and MUC5ac mRNA levels (FIG. 10c) continued to increase and significant mucus production was observed in lung tissue (FIG. 10c), these levels were similar to that induced by IL-13 (data not shown). mRNA was purified with an RNAeasy Plus mini kit (Qiagen) and cDNA was synthesized using Sprint Power Script Double Preprimed 96 kit (Clontech). Gene expression was measured by TaqMan® real-time PCR (Applied Biosystems) following the manufacturer's protocols. The probe sets were obtained from Applied Biosystems as TaqMan® Gene Expression Assays. Taqman reactions contained either the reference gene GAPDH or the genes of interest, IL-5, IL-13, Muc5ac, Gob-5, or mast cell protease-1.

Given the expression of T1/ST2, on TH2 lymphocytes and macrophages, and that repeated IL-33 induced a TH2 response in spleen, the cellular, in particular lymphocyte, subtypes in lung tissue were examined following multiple doses of IL-33. A similar significant dose and time dependent increase in total lung cells were observed (FIG. 10b) as shown for BAL (FIG. 10a). Among the lung lymphocyte population, dose related increases in CD4 (FIG. 10d), and CD4/T1/ST2 positive TH2 cells (FIG. 10e) were found. In addition, increases in macrophages, eosinophils and neutrophils in lung tissue (FIG. 10b) were confirmed.

IL-33 is described as a potent activator of mast cells but studies to date have been performed in vitro (FIG. 8). The effects of IL-33 on local activation in vivo were investigated. To this end, mouse mast cell protease-1 (mMCP-1) levels in serum were measured; elevated serum mMCP-1 has been shown to correlate with mucosal mast cell activation. Similarly, IL-33 was found to induce a significant dose dependent increase in serum mMCP-1 (FIG. 10f) suggesting repeated mast cell activation in vivo, this was not observed with IL-13.

### Example 12

### IL-33-Induced AHR is not CD4 Dependent

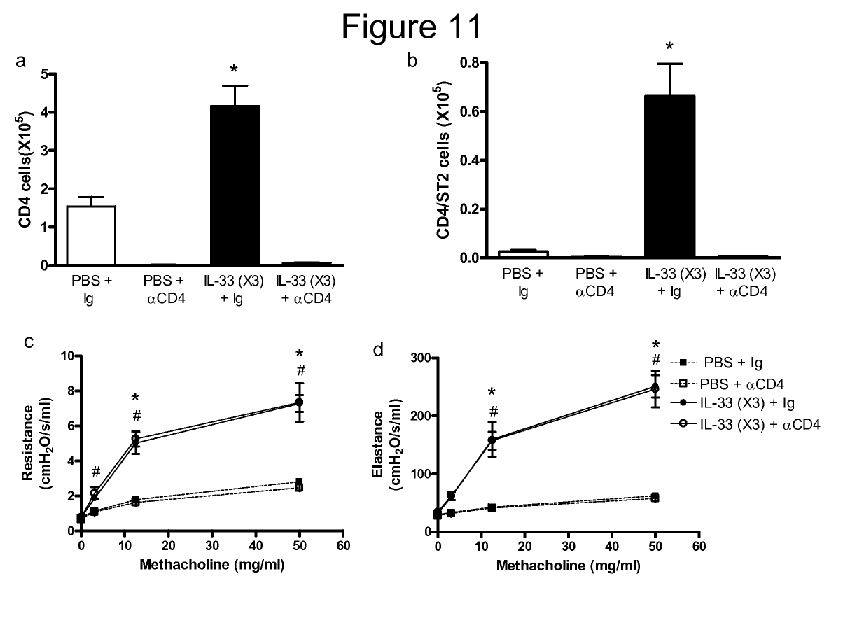

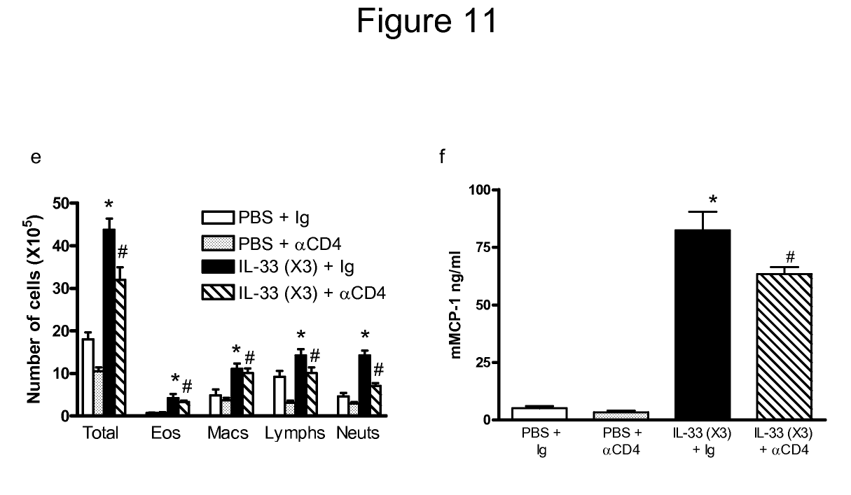

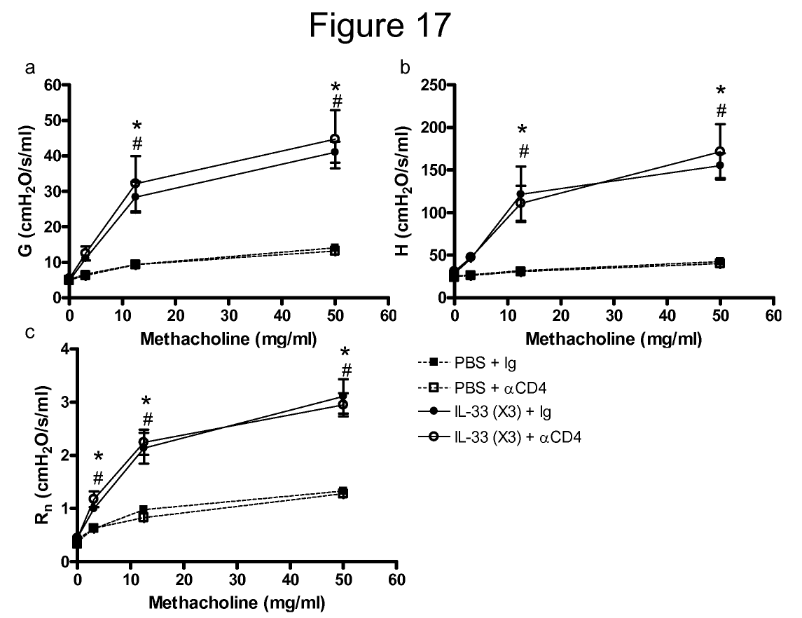

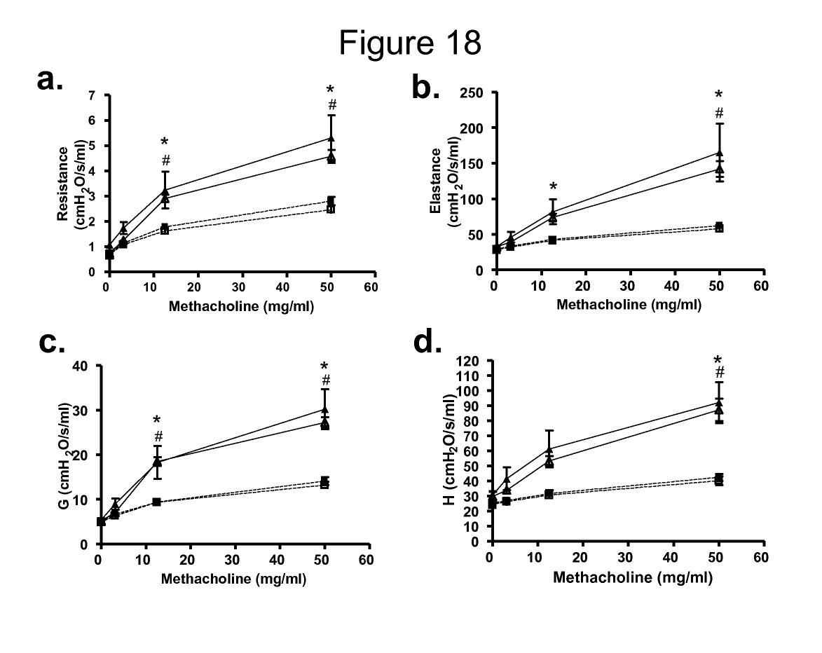

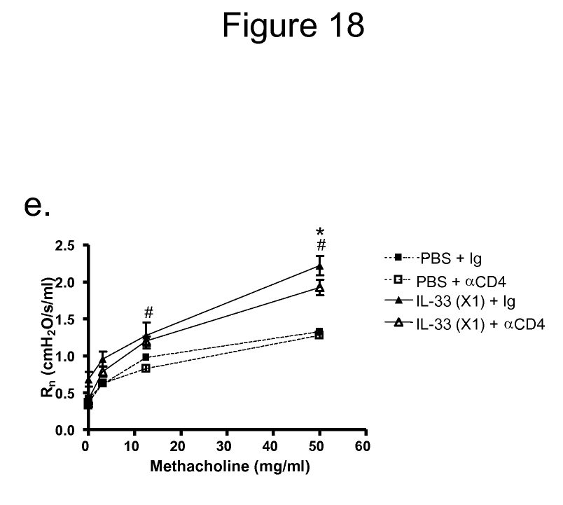

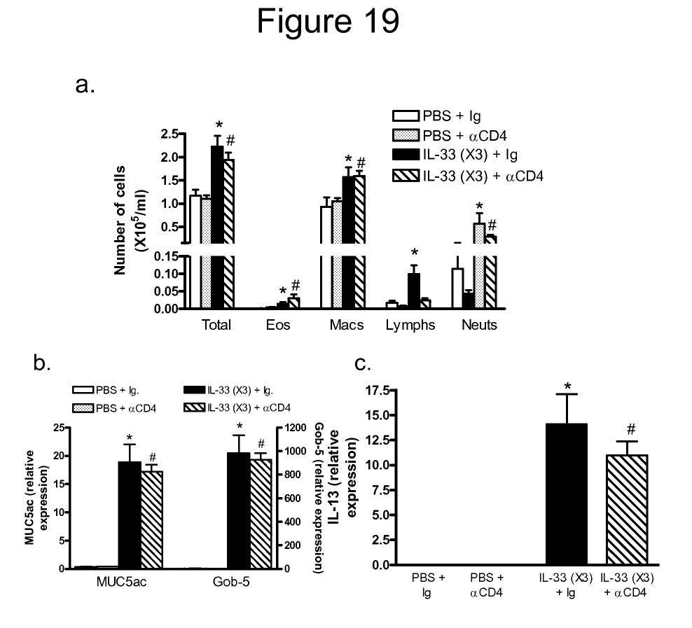

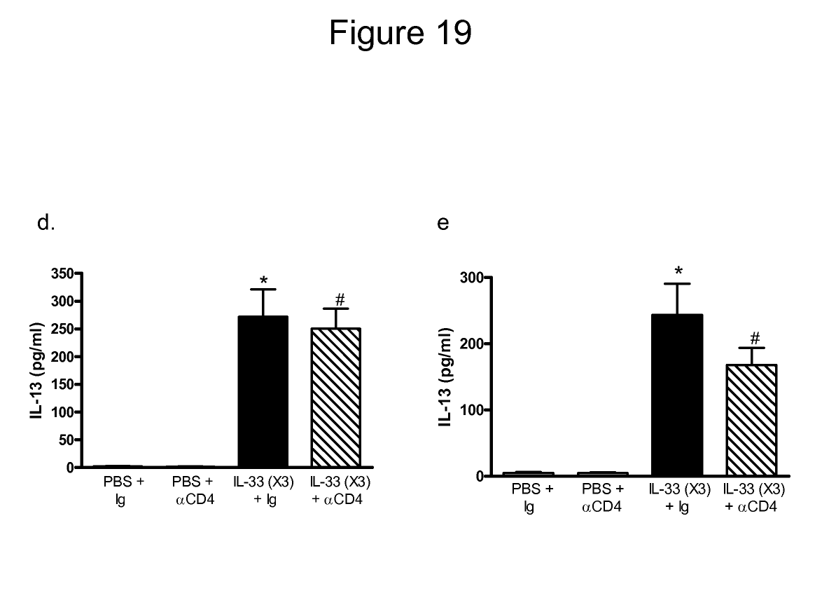

Mechanisms underlying IL-33 induced AHR and inflammation were investigated. A significant recruitment of CD4 and CD4/T1ST2 TH2 cells into the lung following IL-33 was hypothesized to contribute to AHR. Therefore, the effect of single and multiple IL-33 challenges in CD4 depleted animals were investigated. Anti-CD4 depleting antibody pretreatment effectively removed CD4 and CD4/T1ST2 TH2 cells from the lungs of mice (FIG. 11 a,b) but surprisingly had no effect on IL-33 induced AHR (3×IL-33: FIGS. 11c,d and FIG. 17 a-c; 1×IL-33: FIG. 18 a-e). Further, both wild type and CD4 depleted mice had comparable degrees of inflammation (FIG. 11 e, FIG. 19 a), mucus production (FIG. 19 b) and TH2 cytokines, IL-4, IL-5 (not shown) and IL-13 (Bal and Lung FIG. 19 c,d respectively) following IL-33 administration.

### Example 13

### IL-33 Induces AHR via a Mast Cell Dependent Mechanism

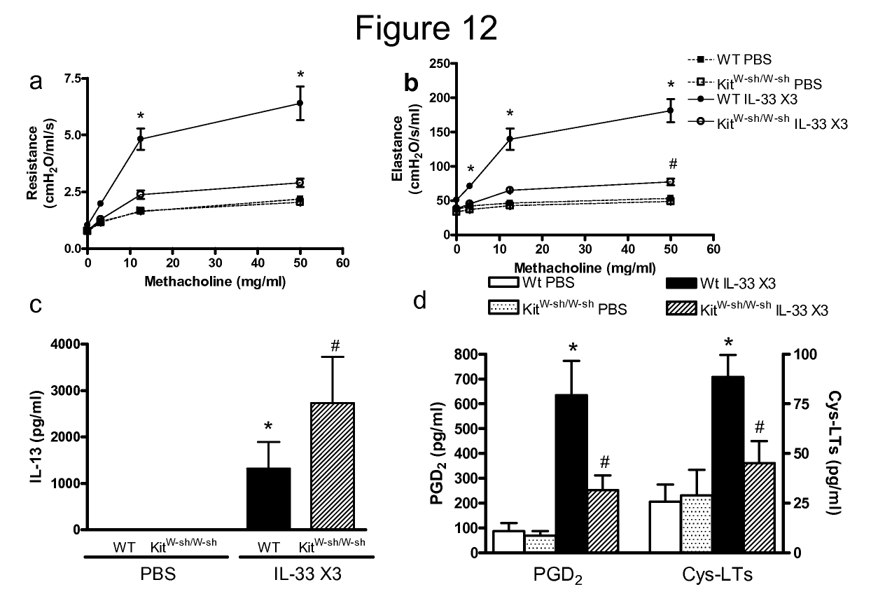

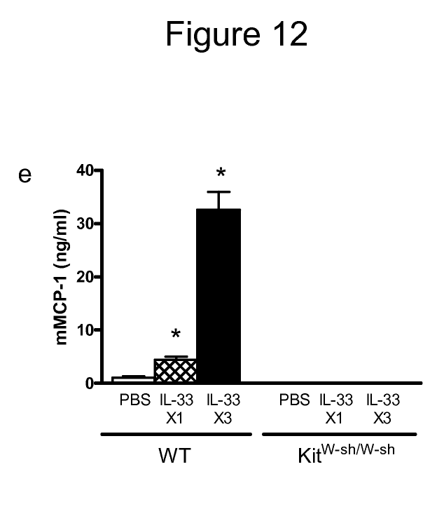

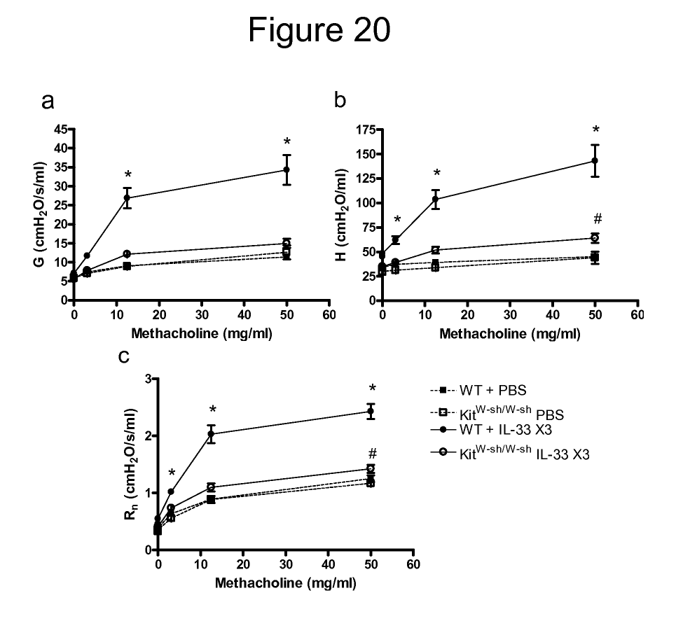

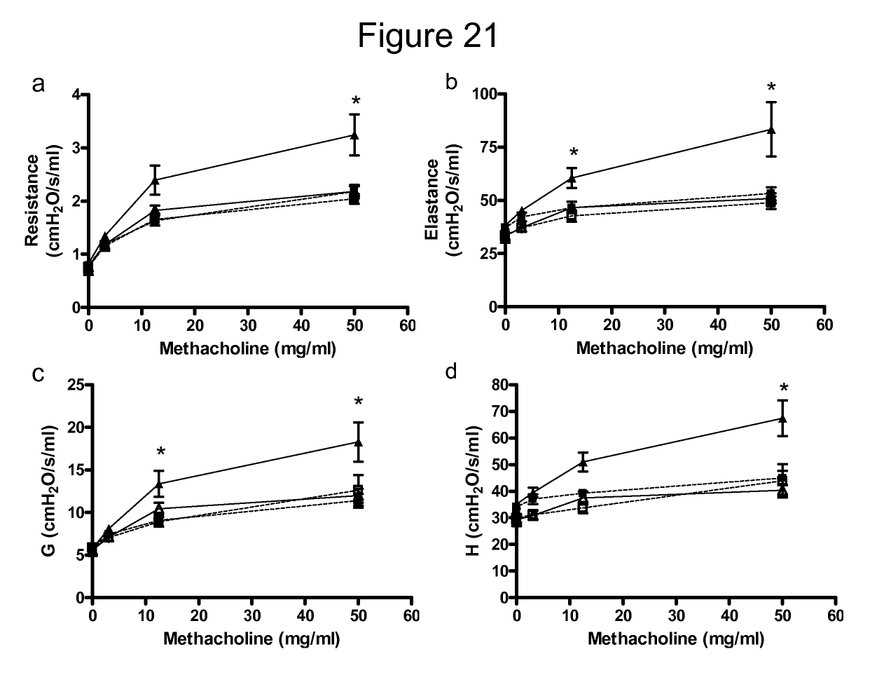

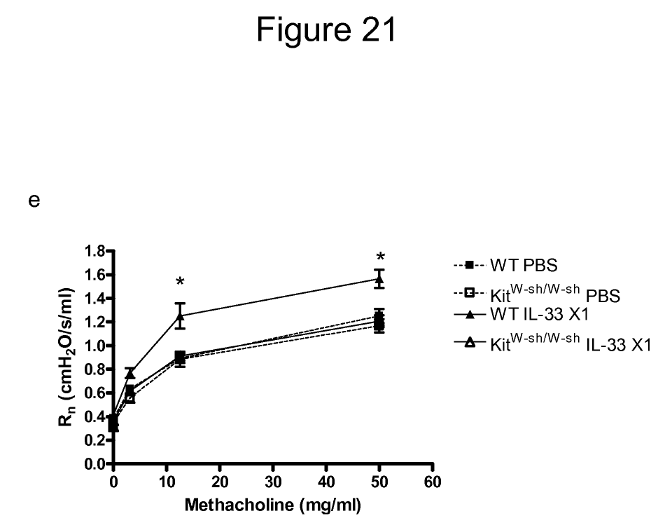

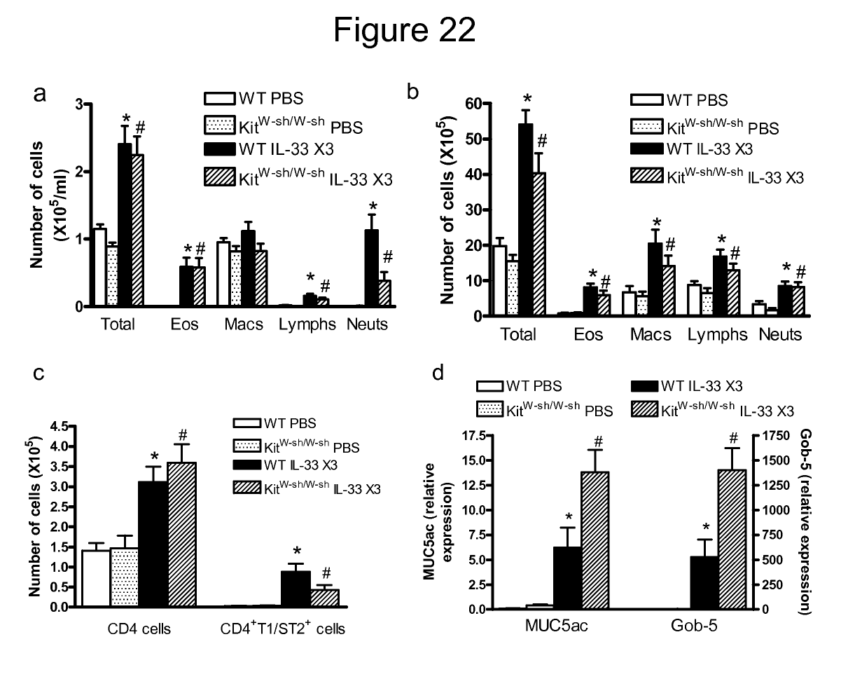

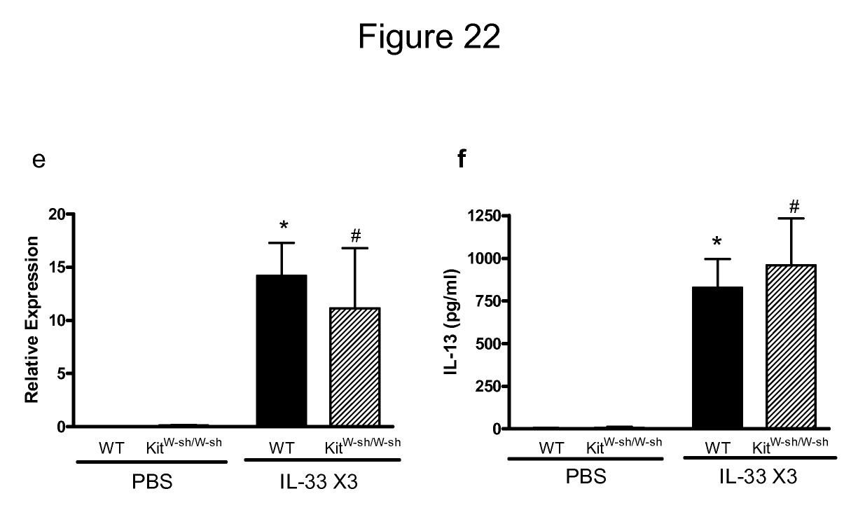

As IL-33 induced significant and repeated mast cell activation in vivo (FIG. 10 f), whether the effects were mast cell mediated was investigated. Thus the responses of IL-33 (1 and 3×) in mast cell deficient mice (Kit^{W-sh}/Kit^{W-sh}) were examined. As seen with BALB/c animals, IL-33 induced a dose related AHR in C57BL/6 wild type controls (FIG. 12 a, b and FIGS. 20, 21). However, Kit^{W-sh}/Kit^{W-sh} mast cell deficient mice were almost completely protected from AHR after both multiple (FIG. 12 a, b and FIG. 20 a-c) and single doses of IL-33 (FIG. 21). Interestingly, mast cell deficiency had no effect on inflammation; no differences in cellular recruitment, including CD4 and CD4/T1ST2 T cells, or the mucin genes GOB-5 and MUC5AC (FIG. 22) were observed. The absence of mast cell activation in Kit^{W-sh}/Kit^{W-sh} mice was confirmed by measuring mMCP-1 levels. IL-33 induced significant elevations of mMCP-1 in C57BL/6 wild type but not Kit^{W-sh}/Kit^{W-sh} mice (FIG. 12 e).

Which of the mast cell mediators contributing to IL-33 induced AHR was investigated. The cytokine IL-13 is produced by both TH2 cells and mast cells and can directly induce AHR in naïve mice. Therefore, the levels of this cytokine in IL-33 treated mice were determined. Significant and comparable levels of IL-13 in both the BAL fluid (FIG. 12 c) and lung tissue (FIG. 22 e, f) of IL-33 treated wild type and Kit^{W-sh}/Kit^{W-sh} mast cell deficient mice were detected, thus indicating no involvement of IL-13 in the AHR.

Other mast cell mediators known to induce bronchial smooth muscle contraction are the eicosanoids, PGD2 and cysteinyl leukotrienes (cysLTs). The levels of these eicosanoids in our mice were examined. IL-33 induced significant increases in both PGD2 and Cys-LTs in the BAL fluid of wild type mice, however these increases were not detected in Kit^{W-sh}/Kit^{W-sh} mast cell deficient mice (FIG. 12 d). These data therefore suggest that IL-33 induced AHR is via mast cell production of PGD2 and cys LTs.

### Example 14

### A role for IL-33 in Allergic Disease

The current results demonstrate that IL-33 is a potent activator of mast cells in vivo, inducing marked bronchoconstriction, TH2 cytokine production and inflammation. These data therefore support a potential role for IL-33 in asthma. Whether allergen challenge would induce IL-33 production in vivo was questioned. BALB/c mice were immunized, (Ovalbumin, OVA), challenged via the airways with OVA and the lung analyzed at various times for IL-33 protein.

Balb/c mice were sensitised intraperitoneally with ovalbumin (OVA; Sigma; 10 μg) in alum (Sigma; 325 μg) on day 0 and 10. On days 19-21, mice received an aerosol challenge of 1% OVA for 30 minutes. Sham mice were sensitised with alum alone, and then challenged through the airways with PBS. Mice were sacrificed 1.5, 3, 6 or 24 hours after the final OVA challenge, and lungs removed for cytokine analysis by ELISA as described above.

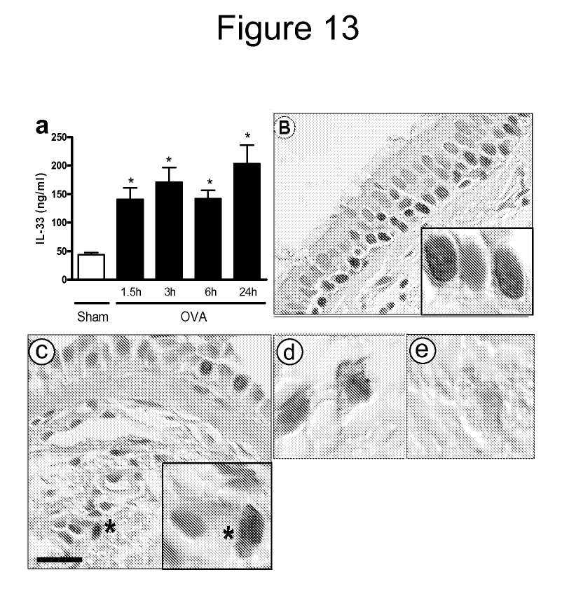

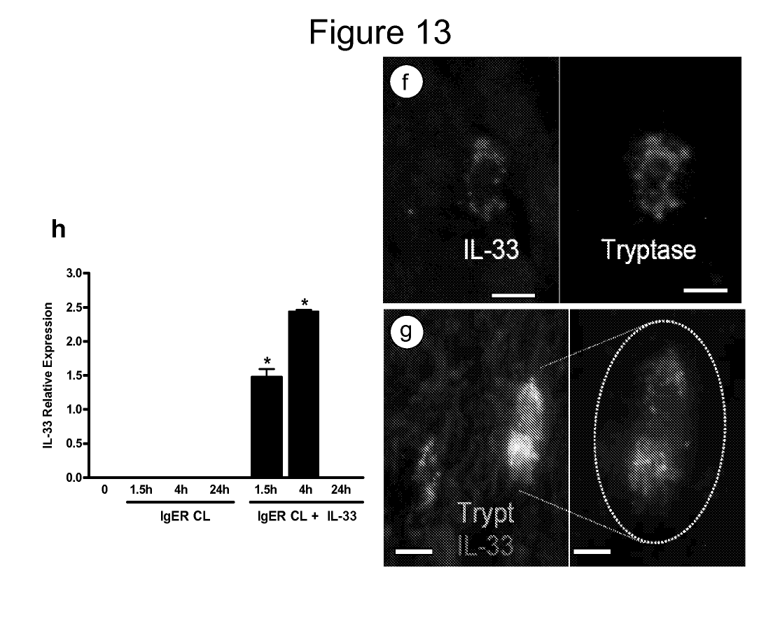

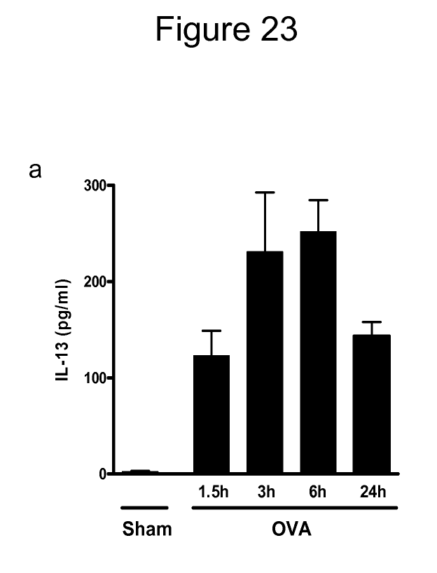

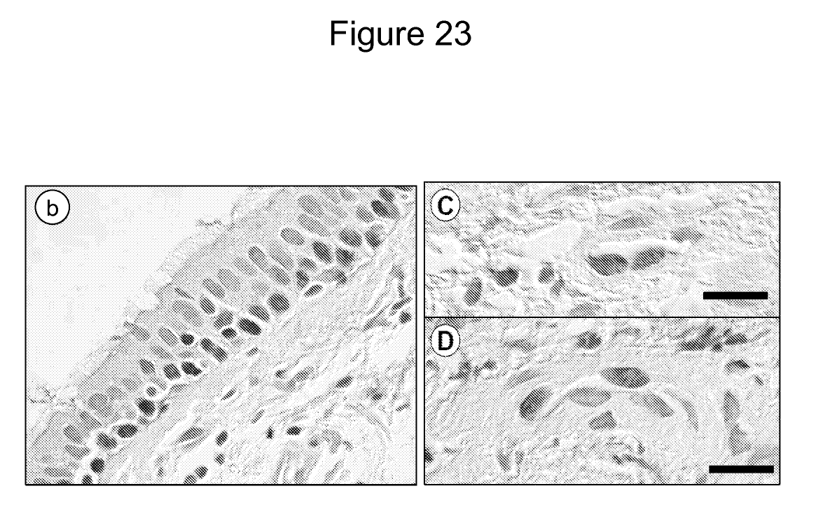

An almost immediate (90 min) and significant induction of IL-33 that was still maintained 24 h after challenge (FIG. 13 a) was observed. Notably, these levels were similar to that of the Th2 cytokine IL-13 following allergen challenge (FIG. 23 a).

The expression of IL-33 in human asthmatic lung biopsies was investigated. The most intense staining in the biopsies was present in the nuclei of structural cells, foremost in epithelial cells (FIG. 13 b and FIG. 23 b) but also in endothelial cells (arrows in FIG. 13 c and FIG. 23 c, d). The epithelial staining was consistent between biopsies and mainly localized to basal cells although consistent but weak nuclei staining within the columnar epithelial cells (i.e. ciliated cells and goblet cells) was observed. Furthermore, endothelial cells of bronchial blood vessels frequently displayed IL-33 immunoreactivity (FIG. 13 c and FIG. 23 c, d). This is consistent with previous reports of an endothelial cell source for IL-33. Of significant interest was the novel observation that a small population of granulated cells, with a mast cell-like morphology, contained IL-33 positive stained granules (FIG. 13 c (inset), d). Importantly, this granule staining was absent in control stained sections (FIG. 13 e). Double immunofluorescence staining for IL-33 and mast cell tryptase (FIG. 13 f) confirmed the presence of IL-33 immunoreactive granules in 10-15% of the total bronchial mast cell population (FIG. 13 g). Increased IL-33 expression has also been detected in asthma patient lung washes collected after segmental allergen challenge (data not shown).

The idea that mast cells are a potential source of IL-33 in asthmatic lung was further examined. To address this, mouse BMMCs were stimulated accordingly and samples were analysed for IL-33 mRNA expression. IL-33 was not detected in IgE receptor activated BMMCs, but a time related increase in mRNA expression in IgE receptor activated+IL-33 stimulated cells was detected. This increase was seen at 90 mins and 4 h but had disappeared by 24 h suggesting de novo synthesis of IL-33 in mast cells (FIG. 13 h).

1. A method of treating an inflammatory disorder comprising:
administering an IL-33 specific binding composition, wherein the composition comprises an antibody, a monomer domain polypeptide, or a multimer domain polypeptide to a subject.

2. The method of claim 1 wherein the inflammatory disorder is asthma.

3. The method of claim 1 wherein the monomer domain comprises a fibronectin scaffold.

4. The method of claim 1 wherein at least one monomer domain of the multimer domain polypeptide comprises a fibronectin scaffold.

5. The method of claim 3 wherein the fibronectin scaffold is a fibronectin 3 scaffold.

6. The method of claim 4 wherein the fibronectin scaffold is a fibronectin 3 scaffold.

7. The method of claim 2 wherein the IL-33 specific binding composition reduces IL-5 levels in lungs of the subject.

8. The method of claim 2 wherein the IL-33 specific binding composition reduces IL-13 levels in lungs of the subject.

9. The method of claim 2 wherein the IL-33 specific binding composition reduces leukotriene levels in lungs of the subject.

10. The method of claim 2 wherein the IL-33 specific binding composition reduces macrophage activation in lungs of the subject.

11. The method of claim 2 wherein the IL-33 specific binding composition reduces macrophage activation in serum of the subject.

12. The method of claim 2 wherein the IL-33 specific binding composition reduces airway hyper-responsiveness of the subject.

13. The method of claim 2 wherein the subject is a human.

14. The method of claim 1 further comprising administering a therapeutic agent which is not an IL-33 specific binding composition.

15. A composition comprising an IL-33 specific polypeptide, wherein the IL-33 specific polypeptide is an antibody, a monomer domain polypeptide, or a multimer domain polypeptide.

16. The composition of claim 15 wherein the polypeptide is a monomer domain polypeptide.

17. The composition of claim 15 wherein the polypeptide is a multimer domain polypeptide.

18. The composition of claim 16 wherein the monomer domain comprises a fibronectin scaffold.

19. The composition of claim 17 wherein the multimer domain comprises at least one monomer domain which comprises a fibronectin scaffold.

20. The composition of claim 18 wherein the fibronectin scaffold is a fibronectin 3 scaffold.

21. The composition of claim 19 wherein the fibronectin scaffold is a fibronectin 3 scaffold.

22. The composition of claim 15 which is sterile.
